(19) United States
(12) Patent Application Publication
(10) Pub. No.: US 2008/0076188 A1
(43) Pub. Date: Mar. 27, 2008

(54) Luminescent compounds

(76) Inventors: Leonid D. Patsenker, Kharkov (UA); Inna G. Yermolenko, Kharkov (UA); Irina A. Fedyunyaeva, Kharkov (UA); Yelena N. Obukhova, Kharkov (UA); Olga N. Semenova, Kharkov (UA); Ewald A. Terpetschnig, Urbana, IL (US)

Correspondence Address: KOLISCH HARTWELL, P.C., 520 SW YAMHILL STREET, Suite 200, PORTLAND, OR, 97204, US

(21) Appl. No.: 11/820,508

(22) Filed: Jun. 19, 2007

Related U.S. Application Data
(60) Provisional application No. 60/814,972, filed on Jun. 19, 2006.

(51) Int. Cl. G01N 21/00; C07C 13/66; C07C 15/27; C07C 15/38; C07C 211/45; C07C 317/00; C07C 43/20; C07C 49/617; C07D 209/56; C07D 219/02; C07D 219/08; C07D 221/06; C07D 221/18; C07D 239/72; C07D 285/14; C07D 307/77; C07D 471/02; C07D 471/04; C07D 471/12; C07D 493/02; C07F 9/28; C07H 21/04; C07K 2/00

(52) U.S. Cl. 436/164; 530/300; 536/23.1; 544/245; 544/246; 544/247; 544/249; 546/100; 546/102; 546/104; 546/37; 546/41; 546/52; 546/76; 546/82; 548/126; 548/420; 548/427; 548/437; 548/440; 549/232; 549/457; 564/308; 568/10; 568/31; 568/326; 568/34; 568/633; 585/26

(57) ABSTRACT

Reporter compounds based on aromatic and heterocyclic compounds, including intermediates used to synthesize the reporter compounds, and methods of synthesizing and using the reporter compounds, where the reporter compounds generally have the structure: 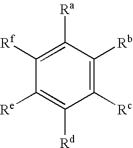
where at least one pair of adjacent substituents (R^{a}, R^{b}), (R^{b}, R^{c}), (R^{c}, R^{d}), (R^{d}, R^{e}), (R^{e}, R^{f}), (R^{f}, R^{a}) is either a substituted cyclic or polycyclic group; or at least one set of three substituents (R^{a}, R^{b}, R^{c}), (R^{b}, R^{c}, R^{d}), (R^{c}, R^{d}, R^{e}), (R^{d}, R^{e}, R^{f}), (R^{e}, R^{f}, R^{a}) is a substituted cyclic or polycyclic group.

### CROSS-REFERENCES TO PRIORITY APPLICATIONS

This application is based upon and claims the benefit under 35 U.S.C. § 119(e) of U.S. Provisional Patent Application Ser. No. 60/814,972, filed Jun. 19, 2006, which is incorporated herein by reference in its entirety for all purposes.

### CROSS-REFERENCES TO RELATED MATERIALS

This application incorporates by reference in their entirety for all purposes all patents, patent applications (published, pending, and/or abandoned), and other patent and nonpatent references cited anywhere in this application. The cross-referenced materials include but are not limited to the following publications: Richard P. Haugland, HANDBOOK OF FLUORESCENT PROBES AND RESEARCH CHEMICALS (6^{th} ed. 1996); JOSEPH R. LAKOWICZ, PRINCIPLES OF FLUORESCENCE SPECTROSCOPY (2^{nd} Ed. 1999); RICHARD J. LEWIS, SR., HAWLEY'S CONDENSED CHEMICAL DICTIONARY (12^{th} ed. 1993).

### TECHNICAL FIELD

The invention relates to compounds based on aromatic and heterocyclic compounds, among others. More particularly, the invention relates to compounds based on aromatic and heterocyclic compounds that are useful as luminescent reporters and long-lifetime labels.

### BACKGROUND

Luminescent compounds may offer researchers the opportunity to use color and light to analyze samples, investigate reactions, and perform assays, either qualitatively or quantitatively. Generally, brighter, more photostable reporters may permit faster, more sensitive and more selective methods to be utilized in such research.

While a calorimetric compound absorbs light, and may be detected by that absorbance, a luminescent compound, or luminophore, is a compound that emits light. A luminescence method, in turn, is a method that involves detecting light emitted by a luminophore, and using properties of that light to understand properties of the luminophore and its environment. Luminescence methods may be based on chemiluminescence and/or photoluminescence, among others, and may be used in spectroscopy, microscopy, immunoassays, and hybridization assays, among others.

Photoluminescence is a particular type of luminescence that involves the absorption and subsequent re-emission of light. In photoluminescence, a luminophore is excited from a low-energy ground state into a higher-energy excited state by the absorption of a photon of light. The energy associated with this transition is subsequently lost through one or more of several mechanisms, including production of a photon through fluorescence or phosphorescence.

Photoluminescence may be characterized by a number of parameters, including extinction coefficient, excitation and emission spectrum, Stokes' shift, luminescence lifetime, and quantum yield. An extinction coefficient is a wavelength-dependent measure of the absorbing power of a luminophore. An excitation spectrum is the dependence of emission intensity upon the excitation wavelength, measured at a single constant emission wavelength. An emission spectrum is the wavelength distribution of the emission, measured after excitation with a single constant excitation wavelength. A Stokes' shift is the difference in wavelengths between the maximum of the emission spectrum and the maximum of the absorption spectrum. A luminescence lifetime is the average time that a luminophore spends in the excited state prior to returning to the ground state. A quantum yield is the ratio of the number of photons emitted to the number of photons absorbed by a luminophore.

Luminescence methods may be influenced by extinction coefficient, excitation and emission spectra, Stokes' shift, and quantum yield, among others, and may involve characterizing fluorescence intensity, fluorescence polarization (FP), fluorescence resonance energy transfer (FRET), fluorescence lifetime (FLT), total internal reflection fluorescence (TIRF), fluorescence correlation spectroscopy (FCS), fluorescence recovery after photobleaching (FRAP), and their phosphorescence analogs, among others.

Luminescence methods have several significant potential strengths. First, luminescence methods may be very sensitive, because modern detectors, such as photomultiplier tubes (PMTs) and charge-coupled devices (CCDs), can detect very low levels of light. Second, luminescence methods may be very selective, because the luminescence signal may come almost exclusively from the luminophore.

Despite these potential strengths, luminescence methods may suffer from a number of shortcomings, at least some of which relate to the nature of the luminescent compound. For example, the luminophore may have an extinction coefficient and/or quantum yield that is too low to permit detection of an adequate amount of light. The luminophore also may have a Stokes' shift that is too small to permit detection of emission light without significant detection of excitation light. The luminophore also may have an excitation spectrum that does not permit it to be excited by wavelength-limited light sources, such as common lasers and arc lamps. The luminophore also may be unstable, so that it is readily bleached and rendered nonluminescent. The luminophore also may have a luminescent lifetime (FLT) that is similar to that of the auto-luminescence of biological and other samples; such autoluminescence is particularly significant at wavelengths below about 600 nm. The luminophore also may be expensive, especially if it is difficult to manufacture.

### SUMMARY

The invention provides luminescent probes and labels based on aromatic and heterocyclic compounds, among others, reactive intermediates used to synthesize these compounds, and methods of synthesizing and using these reporter compounds, among others. These compounds may have luminescent lifetimes in the order of 4 ns-40 ns.

The luminescent compounds relate generally to the following structure: 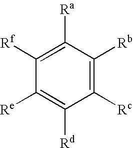
wherein

each R^{a}—R^{f} is independently selected from the group consisting of H, alkyl, alkoxy, amino, alkylamino, dialkylamino, alkenyl, alkynyl, aryl, halogen, sulfo, carboxy, formyl, acetyl, formylmethyl, sulfate, phosphate, phosphonate, ammonium, alkylammonium, cyano, nitro, azido, heterocyclic, substituted heterocyclic, reactive aliphatic and reactive aromatic groups and

wherein at least one pair of adjacent substituents (R^{a}, R^{b}), (R^{b}, R^{c}), (R^{c}, R^{d}), (R^{d}, R^{e}), (R^{e}, R^{f}), (R^{f}, R^{a}) is either a substituted cyclic or polycyclic group W¹, W², W³, W⁴, W⁵, W⁶, W⁷, W⁸, W⁹; or

wherein at least one set of three substituents (R^{a}, R^{b}, R^{c}), (R^{b}, R^{c}, R^{d}), (R^{c}, R^{d}, R^{e}), (R^{d}, R^{e}, R^{f}), (R^{e}, R^{f}, R^{a}) is a substituted cyclic or polycyclic group that is represented by the group consisting of W⁹, W¹⁰, W¹¹, W¹²:

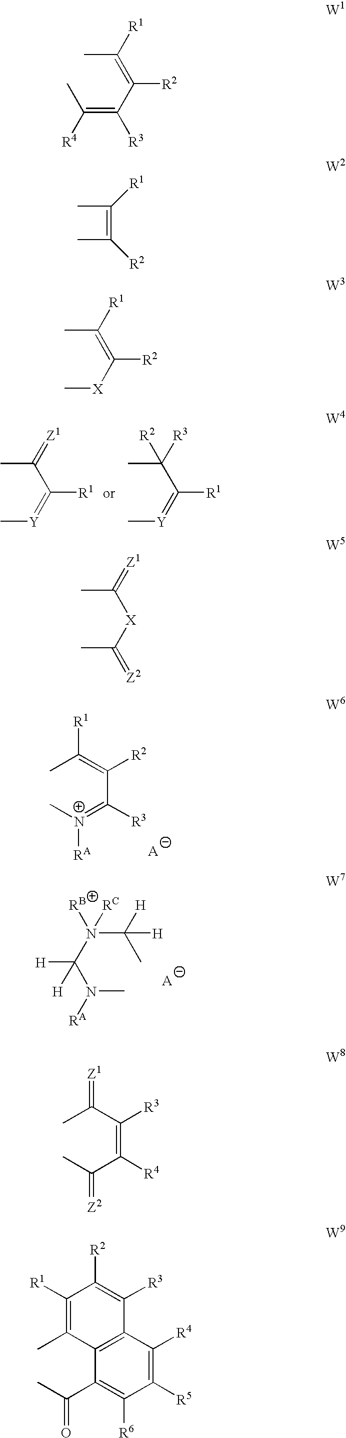

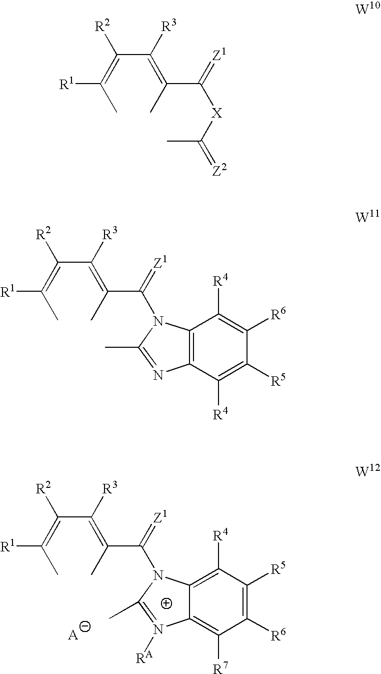

The substituents R^{a}-R^{f}, R¹-R⁷, R^{A}-R^{C}, Z¹, Z², X, Y and A⁻ are defined in the Detailed Description below. The disclosed compounds may include a reactive group and/or a carrier. Alternatively, or in addition, the substituents may be chosen so that the compound is photoluminescent and has a luminescent lifetime in the order of 4 ns or higher.

The disclosed methods relate generally to the synthesis and/or use of reporter compounds for fluorescence lifetime or fluorescence polarization based applications especially those compounds described above.

The nature of the invention will be understood more readily after consideration of the drawings, chemical structures, and detailed description that follow.

### BRIEF DESCRIPTION OF THE DRAWINGS

FIG. 1 is a plot of the absorption and emission spectra of compound 2b in ethanol.

FIG. 2 is a plot of the absorption and emission spectra of compound 3 in water.

FIG. 3 is a plot of the absorption and emission spectra of compound 6 in water.

FIG. 4 is a plot of the absorption and emission spectra of compound 16 in water.

FIG. 5 is a plot of the absorption and emission spectra of compound 18 in water.

FIG. 6 is a plot of the absorption and emission spectra of compound 21 in water.

FIG. 7 is a plot of the absorption and emission spectra of compound 23 in water.

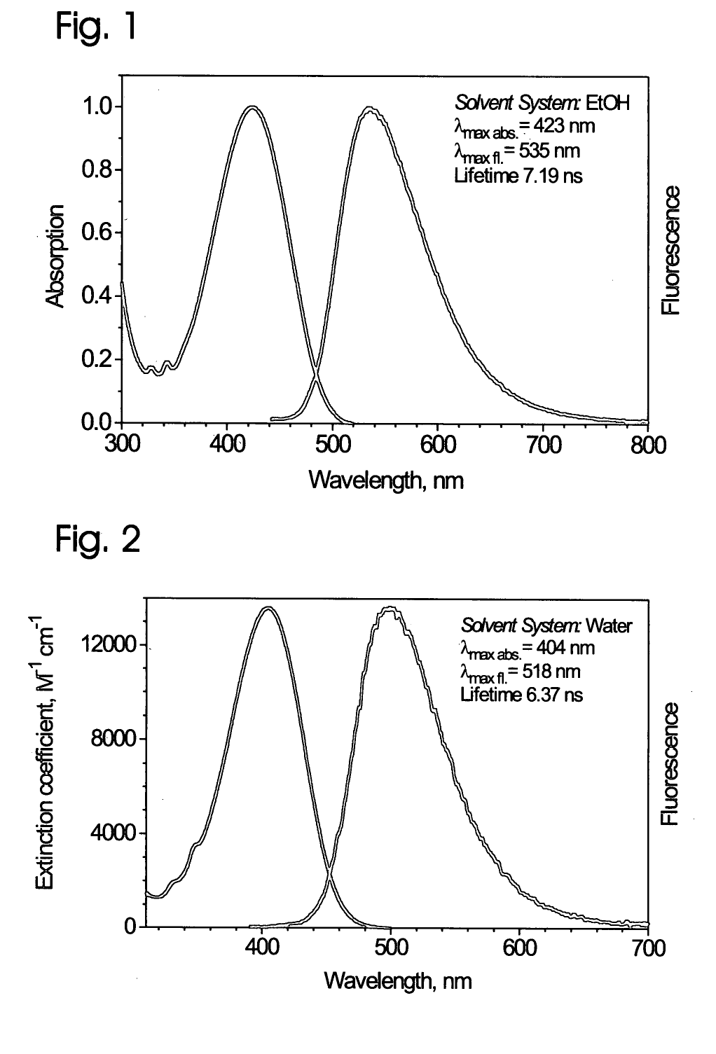

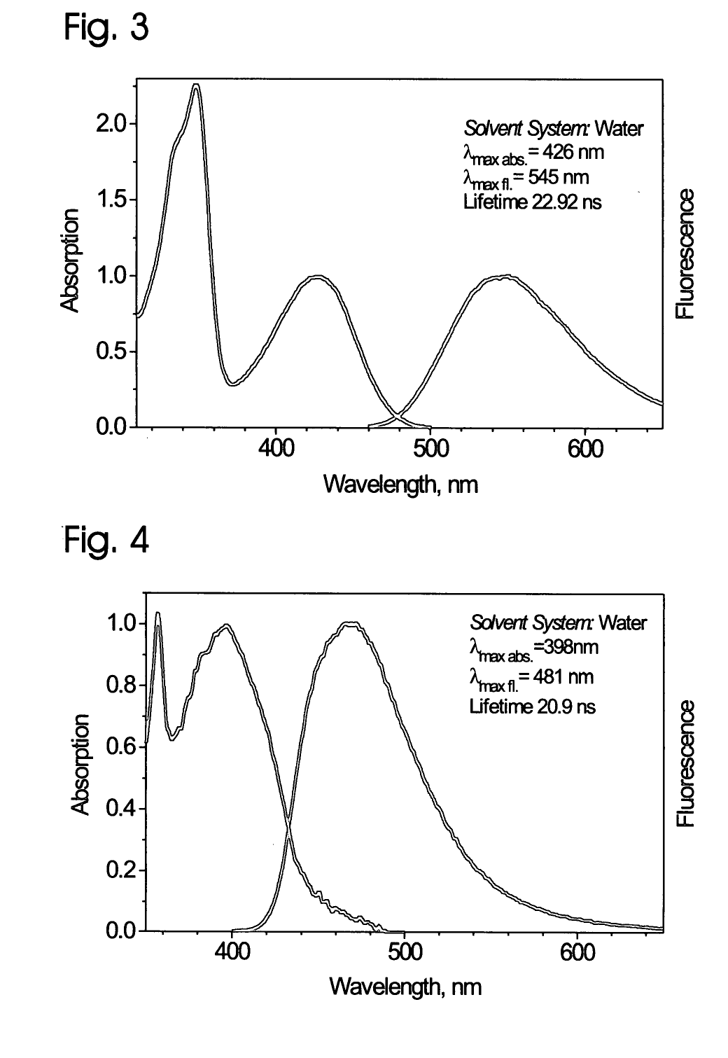

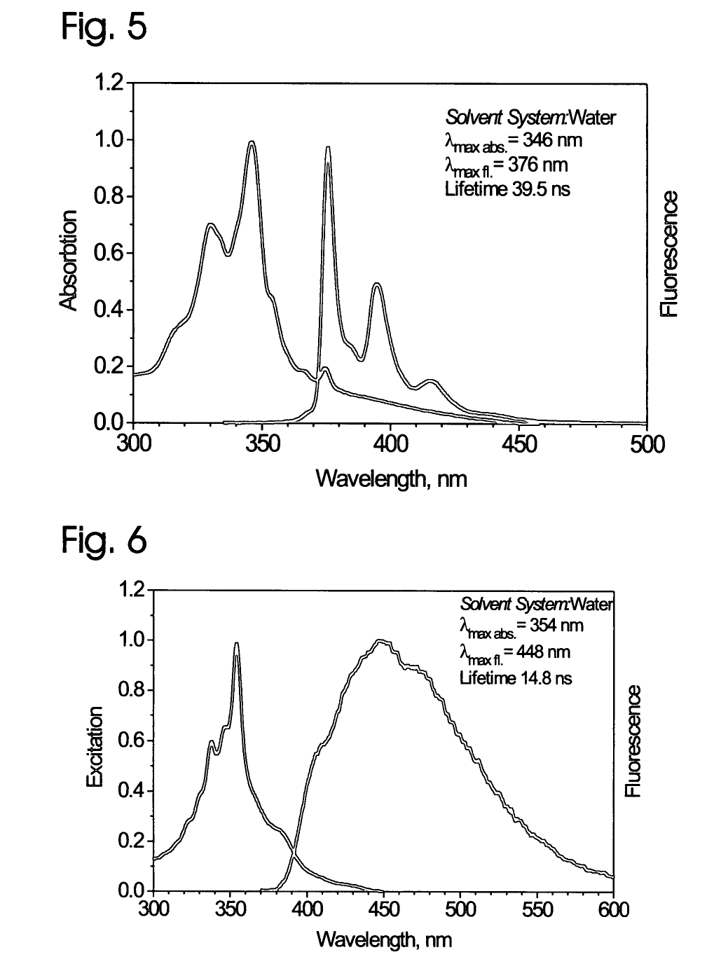

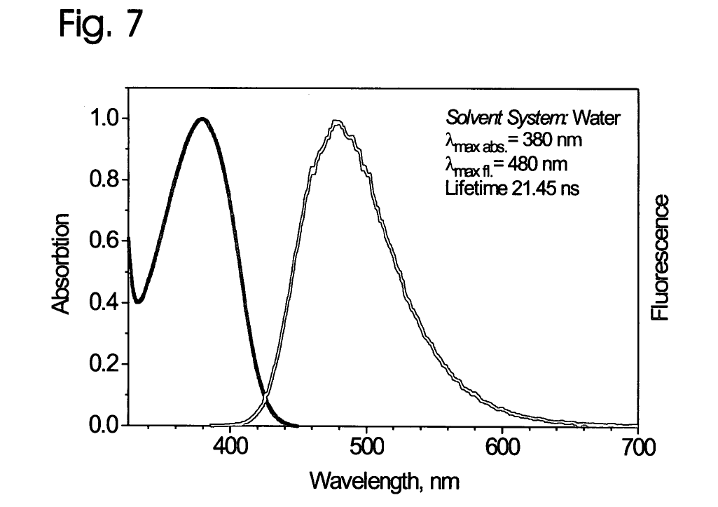

### ABBREVIATIONS

The following abbreviations, among others, may be used in this application:

| Abbreviation | Definition |
|---|---|
| abs | absorption |
| BSA | bovine serum albumin |
| Bu | butyl |
| DCC | dicyclohexylcarbodiimide |
| DIPEA | N,N-diisopropylethylamine |
| DMF | dimethylformamide |
| DMSO | dimethylsulfoxide |
| D/P | dye-to-protein ratio |
| Et | ethyl |
| fl | fluorescence |
| FLT | fluorescence lifetime |
| g | grams |
| h | hours |
| HSA | human serum albumin |
| L | liters |
| Lit. | literature |
| m | milli (10⁻³) |
| M | molar |
| Me | methyl |
| mol | moles |
| M.P. | melting point |
| n | nano (10⁻⁹) |
| ns | nanosecond(s) |
| NHS | N-hydroxysuccinimide |
| NIR | near infrared region |
| PB | phosphate buffer |
| Ph | Phenyl |
| ps | picosecond(s) |
| Prop | propyl |
| TSTU | O-(N-succinimidyl)-N,N,N′,N′-tetramethyluronium |
| | tetrafluoroborate |
| s | second(s) |
| λₘₐₓ(abs) | absorption maximum |
| λₘₐₓ(fl) | emission maximum |
| μ | micro (10⁻⁶) |

### DETAILED DESCRIPTION OF THE INVENTION

The invention relates generally to luminescent compounds having luminescent lifetimes in order of 4 ns and higher and their synthetic precursors, and to methods of synthesizing and using such compounds. These compounds may be useful in both free and conjugated forms, as probes or as labels. This usefulness may reflect in part enhancement of one or more of the following: fluorescence lifetime, fluorescence polarization, quantum yield, Stokes' shift, and photostability.

The remaining discussion includes (1) an overview of structures, (2) an overview of synthetic methods, and (3) a series of illustrative examples.

Overview of Structures

The luminescent reporter compounds may be generally described by the following structure:

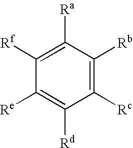

Each R^{a}-R^{f} substituent is independently selected from the group consisting of H, alkyl, alkoxy, amino, alkylamino, dialkylamino, alkenyl, alkynyl, aryl, halogen, sulfo, carboxy, formyl, acetyl, formylmethyl, sulfate, phosphate, phosphonate, ammonium, alkylammonium, cyano, nitro, azido, heterocyclic, substituted heterocyclic, reactive aliphatic and reactive aromatic groups.

In one aspect of the disclosed compounds, at least one pair of adjacent substituents (R^{a}, R^{b}), (R^{b}, R^{c}), (R^{c}, R^{d}), (R^{d}, R^{e}), (R^{e}, R^{f}), (R^{f}, R^{a}) is either a substituted cyclic or polycyclic group W¹, W², W³, W⁴, W⁵, W⁶, W⁷, W⁸, W⁹, as defined below.

In another aspect of the disclosed compounds, at least one set of three substituents (R^{a}, R^{b}, R^{c}), (R^{b}, R^{c}, R^{d}), (R^{c}, R^{d}, R^{e}), (R^{d}, R^{e}, R^{f}), (R^{e}, R^{f}, R^{a}) is a substituted cyclic or polycyclic group that is represented by the group consisting of W⁹, W¹⁰, W¹¹, W¹², as defined below.

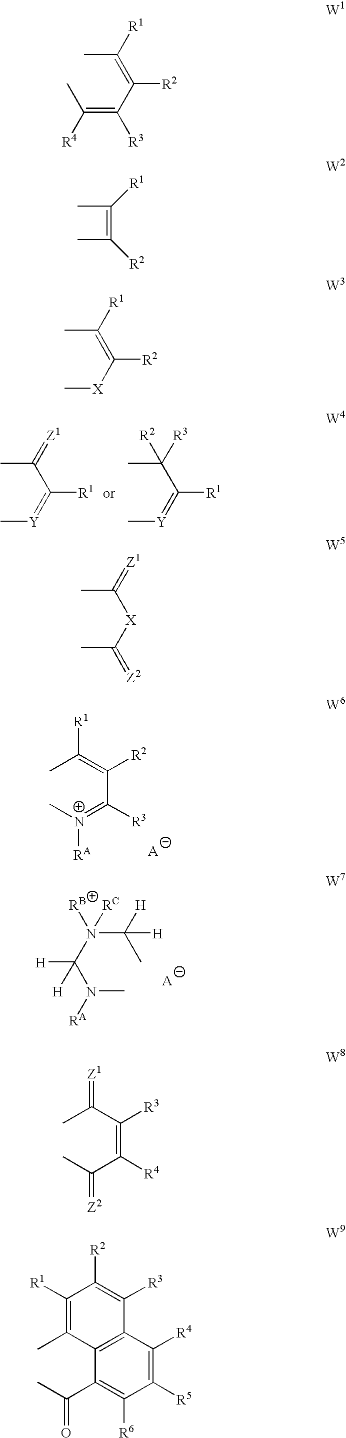

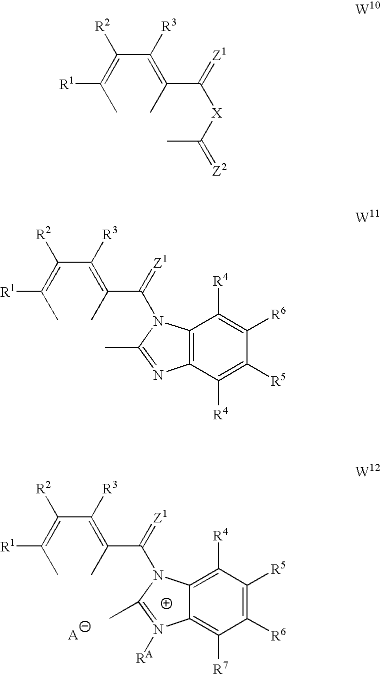

For each of W¹—W¹², each R¹-R⁷ is independently selected from the group consisting of H, alkyl, alkoxy, amino, alkylamino, dialkylamino, alkenyl, alkynyl, aryl, halogen, sulfo, carboxy, formyl, acetyl, formylmethyl, sulfate, phosphate, phosphonate, ammonium, alkylammonium, cyano, nitro, azido, heterocyclic, substituted heterocyclic, reactive aliphatic and reactive aromatic groups.

The X moiety is selected from the group consisting of C(R^{B})(R^{C}), O, S, Se, N—R^{A}.

The Y moiety is selected from the group consisting of CR^{A}, N, ⁺N—R^{A}, O⁺, S⁺; where R^{A} is selected from H, aliphatic groups, alicyclic groups, alkylaryl groups, aromatic groups, -L-S_{c}, -L-R^{x}, and -L-R^{±}, among others.

Each of Z¹ and Z² is independently selected from the group consisting of ═O, ═S, ═Se, ═Te, ═N—R^{A}, and ═C(R^{B})(R^{C}); where R^{B} and R^{C} are independently selected from H, aliphatic groups, alicyclic groups, alkylaryl groups, aromatic groups, -L-S_{c}, -L-R^{x}, -L-R^{±}, among others, or R^{B} and R^{C}, taken in combination, form a cyclic group.

L is a covalent linkage that is linear or branched, cyclic or heterocyclic, saturated or unsaturated, having 1-20 nonhydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen or nitrogen-platinum bonds, or aromatic or heteroaromatic bonds.

The R^{x} moiety is a reactive group. The S_{c} moiety is a conjugated substance. R^{±} is an ionic group, and A⁻ is a biologically compatible and synthetically accessible anion.

In one aspect of the disclosed compounds, the compounds exhibit a luminescence lifetime in the order of 4 nanoseconds (ns) or longer.

The particular substituents on the substituted rings may be chosen quite broadly, and may include any of the various components listed above, in various combinations, among other configurations and substituents.

Reporter Compounds

The compounds disclosed herein may be particularly useful for fluorescence lifetime and fluorescence polarization based applications and methods, as discussed below.

Reactive Groups (R^{x}).

The substituents on the disclosed compounds may include one or more reactive groups, where a reactive group generally is a group capable of forming a covalent attachment with another molecule or substrate. Such other molecules or substrates may include proteins, carbohydrates, nucleic acids, and plastics, among others. Reactive groups (R^{x}) vary in their specificity, and may preferentially react with particular functional groups and molecule types. Thus, reactive compounds generally include reactive groups chosen preferentially to react with functional groups found on the molecule or substrate with which the reactive compound is intended to react.

The compounds of the invention are optionally substituted, either directly or via a substituent, by one or more chemically reactive functional groups that may be useful for covalently attaching the compound to a desired substance. Each reactive group R^{x}, may be bound to the compound directly by a single covalent bond (—R^{x}), or may be attached via a covalent spacer or linkage, -L-, and may be depicted as -L-R^{x}.

The reactive group (—R^{x}) of the invention may be selected from the following functional groups, among others: activated carboxylic esters, acyl azides, acyl halides, acyl halides, acyl nitrites, acyl nitriles, aldehydes, ketones, alkyl halides, alkyl sulfonates, anhydrides, aryl halides, azindines, boronates, carboxylic acids, carbodiimides, diazoalkanes, epoxides, haloacetamides, halotriazines, imido esters, isocyanates, isothiocyanates, maleimides, phosphoramidites, silyl halides, sulfonate esters, and sulfonyl halides.

The following reactive functional groups (—R^{x}), among others, are particularly useful for the preparation of labeled molecules or substances, and are therefore suitable reactive functional groups for the purposes of the reporter compounds:
a) N-hydroxysuccinimide esters, isothiocyanates, and sulfonylchlorides, which form stable covalent bonds with amines, including amines in proteins and amine-modified nucleic acids;
b) Iodoacetamides and maleimides, which form covalent bonds with thiol-functions, as in proteins;
c) Carboxyl functions and various derivatives, including N-hydroxybenztriazole esters, thioesters, p-nitrophenyl esters, alkyl, alkenyl, alkynyl, and aromatic esters, and acyl imidazoles;
d) Alkylhalides, including iodoacetamides and chloroacetamides;
e) Hydroxyl groups, which can be converted into esters, ethers, and aldehydes;
f) Aldehydes and ketones and various derivatives, including hydrazones, oximes, and semicarbozones;
g) Isocyanates, which may react with amines;
h) Activated C═C double-bond-containing groups, which may react in a Diels-Alder reaction to form stable ring systems under mild conditions;
i) Thiol groups, which may form disulfide bonds and react with alkylhalides (such as iodoacetamide);
j) Alkenes, which can undergo a Michael addition with thiols, e.g., maleimide reactions with thiols;
k) Phosphoramidites, which can be used for direct labeling of nucleosides, nucleotides, and oligonucleotides, including primers on solid or semi-solid supports;
l) Primary amines that may be coupled to variety of groups including carboxyl, aldehydes, ketones, and acid chlorides, among others; and
m) Boronic acid derivatives that may react with sugars.
   R Groups

The R moieties associated with the various substituents of Z may include any of a number of groups, as described above, including but not limited to aliphatic groups, alicyclic groups, aromatic groups, and heterocyclic rings, as well as substituted versions thereof.

Aliphatic groups may include groups of organic compounds characterized by straight- or branched-chain arrangement of the constituent carbon atoms. Aliphatic hydrocarbons comprise three subgroups: (1) paraffins (alkanes), which are saturated and comparatively unreactive; (2) olefins (alkenes or alkadienes), which are unsaturated and quite reactive; and (3) acetylenes (alkynes), which contain a triple bond and are highly reactive. In complex structures, the chains may be branched or cross-linked and may contain one or more heteroatoms (such as polyethers and polyamines, among others).

As used herein, “alicyclic groups” include hydrocarbon substituents that incorporate closed rings. Alicyclic substituents may include rings in boat conformations, chair conformations, or resemble bird cages. Most alicyclic groups are derived from petroleum or coal tar, and many can be synthesized by various methods. Alicyclic groups may optionally include heteroalicyclic groups, that include one or more heteroatoms, typically nitrogen, oxygen, or sulfur. These compounds have properties resembling those of aliphatics and should not be confused with aromatic compounds having the hexagonal benzene ring. Alicyclics may comprise three subgroups: (1) cycloparaffins (saturated), (2) cycloolefins (unsaturated with two or more double bonds), and (3) cycloacetylenes (cyclynes) with a triple bond. The best-known cycloparaffins (sometimes called naphthenes) are cyclopropane, cyclohexane, and cyclopentane; typical of the cycloolefins are cyclopentadiene and cyclooctatetraene. Most alicyclics are derived from petroleum or coal tar, and many can be synthesized by various methods.

Aromatic groups may include groups of unsaturated cyclic hydrocarbons containing one or more rings. A typical aromatic group is benzene, which has a 6-carbon ring formally containing three double bonds in a delocalized ring system. Aromatic groups may be highly reactive and chemically versatile. Most aromatics are derived from petroleum and coal tar. Heterocyclic rings include closed-ring structures, usually of either 5 or 6 members, in which one or more of the atoms in the ring is an element other than carbon, e.g., sulfur, nitrogen, etc. Examples include pyridine, pyrole, furan, thiophene, and purine. Some 5-membered heterocyclic compounds exhibit aromaticity, such as furans and thiophenes, among others, and are analogous to aromatic compounds in reactivity and properties.

Any substituent of the compounds of the invention, including any aliphatic, alicyclic, or aromatic group, may be further substituted one or more times by any of a variety of substituents, including without limitation, F, Cl, Br, I, carboxylic acid, sulfonic acid, CN, nitro, hydroxy, phosphate, phosphonate, sulfate, cyano, azido, amine, alkyl, alkoxy, trialkylammonium or aryl. Aliphatic residues can incorporate up to six heteroatoms selected from N, O, S. Alkyl substituents include hydrocarbon chains having 1-22 carbons, more typically having 1-6 carbons, sometimes called “lower alkyl”.

As described in International Publication No. WO01/11370, the substitution of sulfonamide groups such as —(CH₂)ₙ—SO₂—NH—SO₂—R, —(CH₂)ₙ—CONH—SO₂—R, —(CH₂)ₙ—SO₂—NH—CO—R, and —(CH₂)ₙ—SO₂NH—SO₃H, where R is aryl or alkyl and n=1-6, may be used to reduce the aggregation tendency of some compounds, and have some positive effects on their photophysical properties.

Where a compound substituent is further substituted by a functional group R^{±} that is ionically charged, such as for example a carboxylic acid, sulfonic acid, phosphoric acid, phosphonate or a quaternary ammonium group, the ionic substituent R^{±} may serve to increase the overall hydrophilicity of the compound.

As used herein, functional groups such as “carboxylic acid,” “sulfonic acid,” and “phosphoric acid” include the free acid moiety as well as the corresponding metal salts of the acid moiety, and any of a variety of esters or amides of the acid moiety, including without limitation alkyl esters, aryl esters, and esters that are cleavable by intracellular esterase enzymes, such as alpha-acyloxyalkyl ester (for example acetoxymethyl esters, among others).

The compounds of the invention are optionally further substituted by a reactive functional group R^{x}, or a conjugated substance S_{c}, as described below.

The compounds of the invention may be depicted in structural descriptions as possessing an overall charge, it is to be understood that the compounds depicted include an appropriate counter ion or counter ions to balance the formal charge present on the compound. Further, the exchange of counter ions is well known in the art and readily accomplished by a variety of methods, including ion-exchange chromatography and selective precipitation, among others.

Carriers and Conjugated Substances S_{c}

The reporter compounds of the invention, including synthetic precursor compounds, may be covalently or noncovalently associated with one or more substances. Covalent association may occur through various mechanisms, including a reactive functional group as described above, and may involve a covalent linkage, -L-, separating the compound or precursor from the associated substance (which may therefore be referred to as -L-S_{c}).

A covalent linkage binds the reactive group R^{x}, the conjugated substance S_{c} or the ionic group R^{±} to the dye molecule, either directly via a single covalent bond which is depicted in the text as —R^{x}, —R^{±}, —S_{c}, or with a combination of stable chemical bonds (-L-), that include single, double, triple or aromatic carbon-carbon bonds; carbon-sulfur bonds, carbon-nitrogen bonds, phosphorus-sulfur bonds, nitrogen-nitrogen bonds, nitrogen-oxygen or nitrogen-platinum bonds, or aromatic or heteroaromatic bonds; -L- includes ether, thioether, carboxamide, sulfonamide, urea, urethane or hydrazine moieties. Preferably, -L- includes a combination of single carbon-carbon bonds and carboxamide or thioether bonds.

Where the substance is associated noncovalently, the association may occur through various mechanisms, including incorporation of the compound or precursor into or onto a solid or semisolid matrix, such as a bead or a surface, or by nonspecific interactions, such as hydrogen bonding, ionic bonding, or hydrophobic interactions (such as Van der Waals forces). The associated carrier may be selected from the group consisting of polypeptides, polynucleotides, polysaccharides, beads, microplate well surfaces, metal surfaces, semiconductor and non-conducting surfaces, nanoparticles, and other solid surfaces.

The associated or conjugated substance may be associated with or conjugated to more than one reporter compound, which may be the same or different. Generally, methods for the preparation of dye-conjugates of biological substances are well-known in the art. See, for example, Haugland et al., MOLECULAR PROBES HANDBOOK OF FLUORESCENT PROBES AND RESEARCH CHEMICALS, Eighth Edition (1996), or G. T. Hermanson, Bioconjugate Techniques, Academic Press, London, (1996), which is hereby incorporated by reference. Typically, the association or conjugation of a chromophore or luminophore to a substance imparts the spectral properties of the chromophore or luminophore to that substance.

Useful substances for preparing conjugates according to the present invention include, but are not limited to, amino acids, peptides, proteins, phycobiliproteins, nucleosides, nucleotides, nucleic acids, carbohydrates, lipids, ion-chelators, biotin, pharmaceutical compounds, nonbiological polymers, cells, and cellular components. The substance to be conjugated may be protected on one or more functional groups in order to facilitate the conjugation, or to insure subsequent reactivity.

Where the substance is a peptide, the peptide may be a dipeptide or larger, and typically includes 5 to 36 amino acids. Where the conjugated substance is a protein, it may be an enzyme, an antibody, lectin, protein A, protein G, hormones, or a phycobiliprotein. The conjugated substance may be a nucleic acid polymer, such as for example DNA oligonucleotides, RNA oligonucleotides (or hybrids thereof), or single-stranded, double-stranded, triple-stranded, or quadruple-stranded DNA, or single-stranded or double-stranded RNA.

Another class of carriers includes carbohydrates that are polysaccharides, such as dextran, heparin, glycogen, starch and cellulose.

Where the substance is an ion chelator, the resulting conjugate may be useful as an ion indicator (calcium, sodium, magnesium, zinc, potassium and other important metal ions) particularly where the optical properties of the reporter-conjugate are altered by binding a target ion. Preferred ion-complexing moieties are crown ethers (U.S. Pat. No. 5,405,957) and BAPTA chelators (U.S. Pat. No. 5,453,517).

The associated or conjugated substance may be a member of a specific binding pair, and therefore useful as a probe for the complementary member of that specific binding pair, each specific binding pair member having an area on the surface or in a cavity which specifically binds to and is complementary with a particular spatial and polar organization of the other. The conjugate of a specific binding pair member may be useful for detecting and optionally quantifying the presence of the complementary specific binding pair member in a sample, by methods that are well known in the art.

Representative specific binding pairs may include ligands and receptors, and may include but are not limited to the following pairs: antigen-antibody, biotin-avidin, biotin-streptavidin, IgG-protein A, IgG-protein G, carbohydrate-lectin, enzyme-enzyme substrate; ion-ion-chelator, hormone-hormone receptor, protein-protein receptor, drug-drug receptor, DNA-antisense DNA, and RNA-antisense RNA.

Preferably, the associated or conjugated substance includes proteins, carbohydrates, nucleic acids, drugs, and nonbiological polymers such as plastics, metallic nanoparticles such as gold, silver and carbon nanostructures among others. Further carrier systems include cellular systems (animal cells, plant cells, bacteria). Reactive dyes can be used to label groups at the cell surface, in cell membranes, organelles, or the cytoplasm.

The compounds of the disclosure may also be linked to small molecules such as amino acids, vitamins, drugs, haptens, toxins, and environmental pollutants, among others. Another important ligand is tyramine, where the conjugate is useful as a substrate for horseradish peroxidase.

Synthesis and Characterization

The synthesis of the disclosed reporter compounds typically, but not exclusively, is achieved in a multi-step reaction. The synthesis of representative dyes and reactive labels are provided in the Examples below. While the syntheses of non-reactive dyes have been previously described, reactive version and conjugates of the present compounds have not been described. The fluorescent properties of representative dyes are given in Table 1.

The lifetime and fluorescent properties of the presently disclosed dyes may be tuned by selection of the substituents present on the ring systems. For example, the naphtalimide ring system with a dimethylamino substituent has a luminescent lifetime of 4.7 ns when the imide nitrogen is substituted with an aromatic phenyl ring (Table 1, 2a) but becomes twice as long when substituted with a hexanoic acid group (Table 1, compound 2b).

### EXAMPLES

This section describes the synthesis of representative dyes of this disclosure. The spectral properties as well as the luminescent lifetimes of representative dyes in various solvents are provided in Table 1 below. The syntheses of selected protein-conjugates and reactive versions (e.g. NHS esters) for the purpose of covalently labeling biomolecules has also been provided.

### Example 1

Phenylimide (1) and 4-carboxyphenylimide of 4-dimethylaminonaphthalic acid (2a) were synthesized according to the method described in USSR Patent No. 1262911.

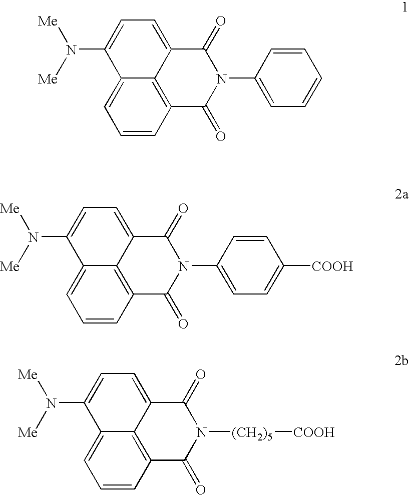

1: Yield 59%. M.P. 269-271° C.

2a: Yield 70%. M.P. 315-318° C.

2b: Yield 20%. M.P. 125-128° C.

### Example 1a

### Synthesis of 6-(5-dimethylamino-1,3-dioxo-2,3-dihydro-1H-benzo[de]-isoquinolin-2-yl)hexanoic acid

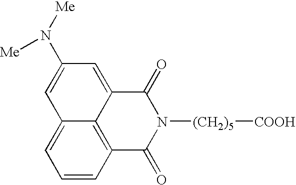

### 2c

2.45 g (0.01 mmol) of 5-nitro-1H,3H-benzo[de]isochromene-1,3-dione and 1.23 g (0.01 mmol) of 6-aminohexanoic acid was alloyed with at 210-220° C. for 35 min. The obtained crude 6-(5-nitro-1,3-dioxo-2,3-dihydro-1H-benzo[de]isoquinolin-2-yl)hexanoic acid was recrystallized from ethanol. Yield 1.7 g (48%). The product was dissolved in 50 mL of ethanol and was added dropwise to the hot solution of 8 g of tin chloride in 9 mL of hydrochloric acid at boiling. The reaction mixture was boiled for 4 h, then poured with water and neutralized with 5% solution of sodium hydrate. Yellow sediment was filtered and purified by a column chromatography (Silica gel, chloroform). Yield 1.1 g of ethyl 6-(5-amino-1,3-dioxo-2,3-dihydro-1H-benzo[de]isoquinolin-2-yl)hexanoate (31.5% counting on 5-nitro-1H,3H-benzo[de]isochromene-1,3-dione). M.p. 108-110° C.

A mixture of 1.8 g (5.08 mmol) of ethyl 6-(5-amino-1,3-dioxo-2,3-dihydro-1H-benzo[de]isoquinolin-2-yl)hexanoate in 9 mL of chloroform was added at 40° C. to a solution of 1.35 g (13.2 mmol) of sodium hydrocarbonate in 6 mL of water. Then 1.3 mL (17.6 mmol) of dimethyl sulfate was added and heated under stirring at 40° C. for 1 h. The reaction mixture was heated at 55-60° C. for 20 min, cooled to RT and dilute with chloroform. The solvent was removed and the residue was suspended in 20 mL of acetic anhydride and heated on the water bath for 40 min. The reaction mixture was poured into water, neutralized with ammonia and extracted with chloroform. The product was column purified (Silica gel, chloroform). The obtained 0.6 g (30%) of ethyl 6-(5-dimethylamino-1,3-dioxo-2,3-dihydro-1H-benzo[de]isoquinolin-2-yl)hexanoate was treated with 0.1 M solution of HCl to yield 0.42 g (75%) of 6-(5-dimethylamino-1,3-dioxo-2,3-dihydro-1H-benzo[de]isoquinolin-2-yl)hexanoic acid. M.p. 111-114° C. ¹H-NMR (200 MHz, DMSO-d₆, δ, ppm): 8.21-7.42 (5H, arom), 3.99 t (2H, α-CH₂, J 7.3 Hz), 3.11 (6H, N(CH₃)₂), 2.21 t (2H, ε-CH₂, J 7.3 Hz), 1.56 m (4H, β,γ-CH₂ J 7.3 Hz), 1.36 m (2H, δ-CH₂, J 7.3 Hz).

### Example 2

Dyes 3, 4 and 5 were synthesized according to procedures described by Patsenker et al. (L. D. Patsenker et al., Tetrahedron, 2000, V. 56, No. 37, P. 7319-7323).

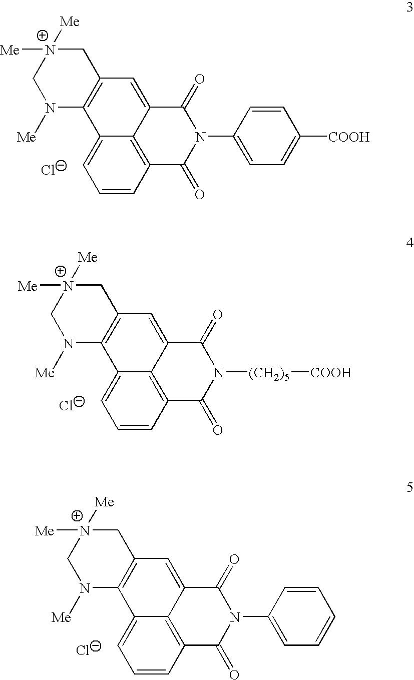

### Synthesis of 5-(4-carboxyphenyl)-9,9,11-trimethyl-4,6-dioxo-5,6,8,9,10,11-hexahydro-4H-isoquino[4,5-α,h]quinazolin-9-ium chloride (3)

Compound 3 was obtained by the same procedure as 5 using 1.44 g (4 mmol) of phenylimide instead of carboxyphenylimide. Yield 0.92 g (51%), yellow solid. M.P. 255-258° C. Found: C, 63.9; H, 4.8; Cl, 7.8; N, 9.4, C₂₄H₂₁ClN₃O₄. Calculated: C, 63.93; H, 4.69; Cl, 7.86; N, 9.32%; IR, νₘₐₓ(KBr) 1715, 1695, 1660, 1600, 1570, 1465, 1404, 1380, 1350, 1280, 1240 cm⁻¹; ¹H-NMR (300 MHz, DMSO-d₆, δ, ppm) 3.32 (6H, s, ⁺N(CH₃)₂), 3.73 (3H, s, 4-NCH₃), 5.01 (2H, s, CH₂), 5.25 (2H, s, CH₂), 7.38-8.67 (8H, m, arom H).

### Example 2a

### Synthesis of 5-(5-carboxypentyl)-8,10,10-trimethyl-4,6-dioxo-5,6,8,9,10,11-hexahydro-4H-isoquino[5,4-fg]quinazolin-10-ium chloride (4a)

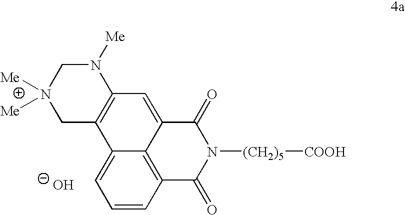

To a solution of 90 mg (0.25 mmol) of 6-(5-dimethylamino-1,3-dioxo-2,3-dihydro-1H-benzo[de]isoquinolin-2-yl)hexanoic acid in 0.4 mL of DMF 0.09 ml of POCl₃ were added dropwise at 40° C. The mixture was heated under stirring at 80° C. for 1.5 h, cooled to RT and poured into ice water. The product was precipitated with isopropyl alcohol and purified by column chromatography on reverse phase (PR-18, H₂O-acetonitrile 5:1, v/v). Yield: 45 mg (40%). M.p. 186-188° C. ¹H-NMR (200 MHz, DMSO-d₆, δ, ppm): 8.39-7.82 (5H, arom), 5.17 s (2H, CH₂), 4.85 s (2H, CH₂), 4.04 t (2H, α-CH₂, J 7.3 Hz), 3.65 s (3H, NCH₃), 3.19 s (6H, ⁺N(CH₃)₂), 1.94 t (2H, ε-CH₂, J 7.3 Hz), 1.54 m (4H, β,γ-CH₂ J 7.3 Hz), 1.26 m (2H, δ-CH₂, J 7.3 Hz).

### Synthesis of 5-(5-carboxypentyl)-9,9,11-trimethyl-4,6-dioxo-5,6,8,9,10,11-hexahydro-4H-isoquino[4,5-g,h]quinazolin-9-ium chloride (4)

To a mixture of 0.177 g (0.5 mmol) of 2b and 0.5 mL (6.5 mmol) of DMF 0.18 mL (2 mmol) of POCl₃. The mixture was heated at 80° C. for 1.5 h, treated with ice, and acetone was added to precipitate the oily product, which was treated with ether to give yellow crystals.

### Synthesis of 9,9,11-trimethyl-4,6-dioxo-5-phenyl-5,6,8,9,10,11-hexahydro-4H-isoquino[4,5-g,h]quinazolin-9-ium chloride (5)

1.26 g (4 mmol) of 4-carboxyphenylimide of 4-dimethylaminonaphthalic acid (2) were dissolved in 5 mL (65 mmol) of DMF, and then 1.7 mL (18 mmol) of POCl₃ were added dropwise at 60-70° C. The mixture was heated with stirring at 100° C. for 25 min, cooled to RT and poured into ice water. The crude product was recrystallized from ethanol to give the 5 (0.86 g, 53%) as a yellow solid, M.P. 235° C. Found: C, 66.3; H, 5.5; Cl, 8.7; N, 10.4, C₂₃H₂₂ClN₃O₂.0.5H₂O. Calculated: C, 66.26; H, 5.56; Cl, 8.50; N, 10.08%. IR, νₘₐₓ(KBr) 1695, 1650, 1600, 1565, 1450, 1400, 1375, 1340 cm⁻¹. ¹H-NMR (300 MHz, DMSO-d₆, δ, ppm) 3.42 (6H, s, ⁺N(CH₃)₂), 3.88 (3H, s, 4-NCH₃), 5.00 (2H, s, CH₂), 5.16 (2H, s, CH₂), 7.42-8.86 (9H, m, arom H).

### Example 3

### Synthesis of 8,10,10-trimethyl-4,6-dioxo-5-phenyl-5,6,8,9,10,11-hexahydro-4H-isoquino[5,4-fg]quinazolin-10-ium hexafluorophosphate (6)

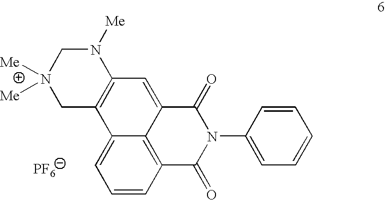

To a mixture of 3.16 g (0.01 mol) of phenylimide 3-dimethylaminonaphthalic acid and 13.8 mL (0.18 mol) of DMF at 30-35° C. 4.1 mL (0.045 mol) of POCl₃ were added dropwise. The mixture was stirred at 80° C. for 30 min, cooled down to RT, and treated with ice. Then LiPF₆ was added and the obtained precipitate was filtered off and dried. Yield 3.10 g (60%). M.P. 259-260° C. ¹H-NMR (300 MHz, δ, ppm,): 8.36 d (1H, H⁷, J 7.3 Hz), 8.18 s (1H, H⁵), 8.14 d (1H, H², J 8.4 Hz), 7.93 dd (1H, H⁶, J₁ 8.3, J₂ 7.4 Hz), 7.59-7.34 m (5H, phenyl), 5.22 s (2H, CH₂), 4.87 s (2H, CH₂), 3.39 s (3H, N—CH₃), 3.22 s (6H, ⁺N(CH₃)₂). Found, %: C, 53.35; H, 4.30; N, 10.99, C₂₃H₂₂F₆N₃O₂P. Calculated, %: C, 53.39; H, 4.26; N, 11.25.

### Example 4

Dyes 7a, 7b, and 8 were synthesized according to Lyubenko et al. (O. N. Lyubenko, et al., Chem. Heterocycl. Compd., Engl. Transl., 2003, No. 4, P. 594).

### Synthesis of intermediate isomeric ethyl 4-dimethylamino-(1a) and ethyl 3-dimethylamino (1b)-10-methyl-7-oxo-7H-benzo[de]pyrazolo[5,1-a]isoquinoline-11-carboxylates

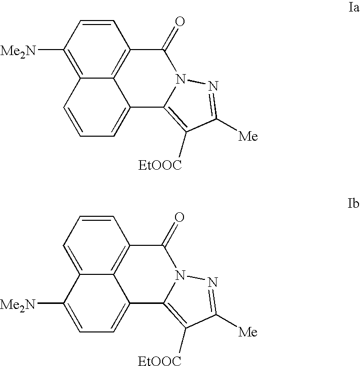

A mixture of 5 mmol of 2-amino-6-dimethylamino-2,3-dihydro-1H-benzo[de]isoquinoline-1,3-dione (O. N. Lyubenko, et al., Chem. Heterocycl. Compd., Engl. Transl., 2003, No. 4, P. 594), 4.5 mL (35 mmol) of acetoacetic acid diethyl ester and 0.01 g (0.057 mmol) p-toluenesulfonic acid was stirred at 130° C. for 4 h under nitrogen atmosphere. The obtained precipitate of hydrazone was filtered off, washed with methanol, water, and dried. Then the hydrazone was refluxed for 1 h in 2.5 mL of DMF and 0.01 g (0.12 mmol) of NaOAc. The precipitate was filtered off, washed with methanol, water, and dried. Isomeric dyes 1a and 1b were separated using a column chromatography (Al₂O₃, benzene). 1a. Yield 18%. M.P. 204-205° C. ¹H-NMR, (200 MHz, DMSO-d₆, δ, ppm): 9.28 (1H, d, J=7.6, 7-H), 8.52 (1H, d, J=8.5, 2-H), 8.38 (1H, d, J=8.5, 5-H), 7.68 (1H, t, J=8.0, 6-H), 7.24 (1H, d, J=8.5, 3-H), 7.2; COOCH₂CH₃), 4.42 (2H, q, J=14.1), 3.19 (6H, s, N(CH₃)₂), 2.50 (3H, s, CH₃), 1.39 (3H, t, J=7.2, COOCH₂CH₃). Found, %: C, 68.70; H, 5.38; N, 11.60, C₂₀H₁₉N₃O₃. Calculated, %: C, 68.77; H, 5.44; N, 12.03. IR (ν, cm⁻¹, KBr): 1680 (C═O carbonyl), 1710 (C═O ester). 1b. Yield 12%. M.P. 194-195° C. ¹H-NMR, (200 MHz, DMSO-d₆, δ, ppm): 9.26 (1H, d, J=8.4, 7-H), 8.68 (1H, d, J=7.3, 2-H), 8.62 (1H, dd., J=8.4; 0.7; 4-H), 7.83 (1H, t, J=7.9, 3-H), 7.18 (1H, d, J=8.5, 6-H), 4.38 (2H, q, J=14.2; 7.1; COOCH₂CH₃), 3.07 (6H, s, N(CH₃)₂), 2.48 (3H, s, CH₃), 1.39 (3H, t, J=7.1, COOCH₂CH₃). Found, %: C 68.71; H, 5.40; N, 11.79, C₂₀H₁₉N₃O₃. Calculated, %: C, 68.77; H, 5.44; N, 12.03. IR (ν, cm⁻¹, KBr): 1680 (C═O carbonyl), 1710 (C═O ester).

General Procedure for the Synthesis of Dyes 7a, 7b, and 8.

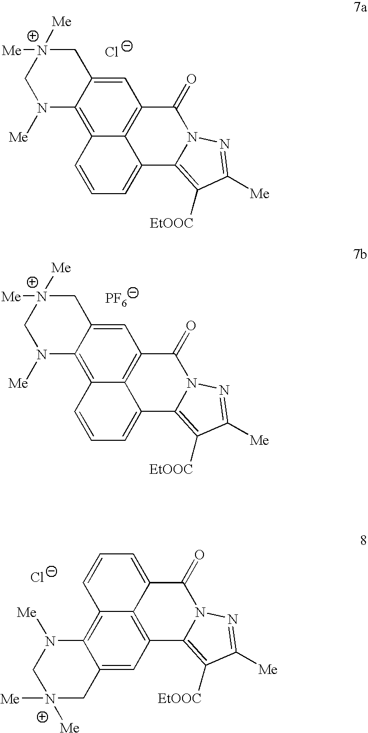

To a mixture of 1 mmol of pyrazole 1a or 1b in 2 mL (26 mmol) of DMF at 60° C. 0.37 mL (4 mmol) of POCl₃ was added dropwise. The mixture was stirred at 100° C. for 3 h in case of 1a and 4 h in case of 1b, cooled down and poured into ice. 13-Acetyl-4,6,6,12-tetramethyl-9-oxo-4,6,7,9-tetrahydro-5H-pyrazolo[5′, 1′: 1,2]isoquino[4,5-gh]quinazoline-6-ium chloride (7a) and 12-acetyl-2,2,4,11-tetramethyl-8-oxo-2,3,4,8-tetrahydro-1H-pyrazolo[1′,5′:2,3]isoquino[4,5-gh]quinazoline-2-ium chloride (8) were precipitated by isopropanol. Chlorides 7a and 13 were recrystallized from ethanol. Crystalline 13-acetyl-4,6,6,12-tetramethyl-9-oxo-4,6,7,9-tetrahydro-5H-pyrazolo-[5′,1′:1,2]isoquino[4,5-gh]quinazoline-6-ium hexafluorophosphate 7b was obtained using 0.15 g (1 mmol) of LiPF₆, and then was column purified (Silochrom C-120, acetonitrile). 7a: Yield 30%. M.P. 251-252° C. (ethanol). ¹H-NMR (200 MHz, DMSO-d₆, δ, ppm): 9.27 (1H, d, J=7.6, 7-H), 8.39 (1H, d J=8.6, 5-H), 8.32 (1H, s 2-H), 7.75 (1H, t J=8.2, 6-H), 5.17 (2H, s CH₂), 5.02 (2H, s CH₂), 4.42 (2H, q, J=14.0; 7.0; COOCH₂CH₃), 3.72 (3H, s, NCH₃), 3.31 (6H, s, N⁺(CH₃)₂), 2.49 (3H, s, CH₃), 1.41 (3H, t, J=7.2, COOCH₂CH₃). Found, %: C, 62.59; H, 5.73; N, 12.23; Cl 7.89. C₂₃H₂₅N₄O₃Cl. Calculated, %: C, 62.65; H, 5.67; N, 12.71; Cl 8.06. IR (ν, cm⁻¹, KBr): 1680 (C═O carbonyl), 1700 (C═O ester). 7b: Yield 45%. M.P. 315-318° C. (acetonitrile). ¹H-NMR (200 MHz, DMSO-d₆, δ, ppm): 1.41 (3H, t, J=7.1, COOCH₂CH₃); 2.52 (3H, s, CH₃); 3.24 (6H, s, N⁺(CH₃)₂); 3.69 (3H, s, NCH₃); 4.44 (2H, q, J=14.1; 7.0; COOCH₂CH₃); 4.93 (2H, s, CH₂); 5.06 (2H, s, CH₂); 7.84 (1H, t, J=8.1, 6-H); 8.45 (1H, s, 2-H); 8.46 (1H, d, J=7.9, 5-H); 9.36 (1H, d, J=7.6, 7-H). Found, %: C, 50.11; H, 4.46; N, 10.54, C₂₃H₂₅N₄O₃ PF₆. Calculated, %: C, 50.18; H, 4.54; N, 10.18. IR (ν, cm⁻¹, KBr): 1700 (C═O carbonyl), 1680 (C═O ester). 8: Yield 34%. M.P. 242-245° C. (ethanol). ¹H-NMR (200 MHz, DMSO-d₆, δ, ppm): 1.41 (3H, t, J=7.2, COOCH₂CH₃); 2.54 (3H, s, CH₃); 3.30 (6H, s, N⁺(CH₃)₂); 3.68 (3H, s, NCH₃); 4.41 (2H, q, J=14.1; 7.2; COOCH₂CH₃); 4.92 (2H, s, CH₂); 5.11 (2H, s, CH₂); 7.91 (1H, t, J=7.9, 3-H); 8.65 (1H, d, J=6.3, 2-H); 8.68 (1H, d, J=8.2, 4-H); 9.16 (1H, s, 7-H). Found, %: C, 62.59; H, 5.72; N, 12.66; Cl 8.23, C₂₃H₂₅N₄O₃Cl. Calculated, %: C, 62.65; H, 5.67; N, 12.71; Cl 8.06. IR (ν, cm⁻¹, KBr): 1650 (C═O carbonyl), 1700 (C═O ester).

### Example 5

13-ethyl-7-oxo-7H-benzo[de]benzo[4,5]imidazo[2,1-a]isoquinolin-13-ium 4-methyl-1-benzenesulfonate (9) was synthesized according to (USSR Patent 493-496).

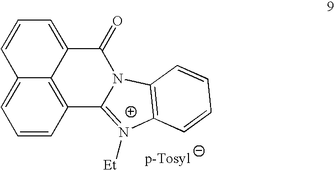

A mixture of 3 g of 1,8-naphthoilene-1′,2′-benzimidazole and 10 g of ethyl p-toluenesulfonate was heated at 200° C. for 15 min, cooled down to 20-30° C. and treated with 70 mL of toluene. The obtained crystalline product was washed with 10 mL of toluene, dried at 70-80° C., and recrystallized from ethanol. M.P. 215-217° C. Found, %: N 5.85; S 6.33. C₂₇H₂₂N₂O₄S. Calculated, %: N 5.95; 6.81.

### Example 5a

### Synthesis of 1,2-dihydrobenzo[cd]indol-2-one (9a)

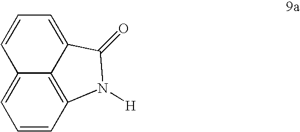

20 g (0.1 mol) of 1H,3H-benzo[de]isochromene-1,3-dione and 9 g (0.129 mol) of hydroxylamine hydrochloride in 500 mL of 2% solution of sodium carbonate were boiled for 3 h. Then 500 mL of 10% sodium carbonate solution were added and heated to boiling. After cooling 17 g of 2-hydroxy-2,3-dihydro-1H-benzo[de]isoquinoline-1,3-dione sodium salt were obtained.

16 g (0.084 mol) of p-toluenesulfonic acid was added to a mixture of 17 g (72 mmol) of 2-hydroxy-2,3-dihydro-1H-benzo[de]isoquinoline-1,3-dione sodium salt in 400 mL of dry benzene, refluxed for 6 h, and the hot mixture was filtered. The obtained 2-(4-methylphenylsulfonyloxy)-1,3-dioxo-2,3-dihydro-1H-benzo[de]isoquinoline (12.85 g, 50%) was suspended in 600 mL of methanol. Then 91 mL of 0.5 N solution of sodium hydroxide in methanol were added. After stirring at RT for 1 h the mixture was neutralized with HCl. The solvent was removed on a rotary evaporator and residue was washed several times with water. Yield: 5.6 g (95%). M.p. 170-172° C. ¹H-NMR (200 MHz, DMSO-d₆, δ, ppm): 10.70 s (1H, NH), 8.17 d (1H, H³, J 8.1 Hz), 8.02 d (1H, H³, J 7.2 Hz), 7.79 t (1H, H³, J 7.8 Hz), 8.17 d (1H, H³, J 8.1 Hz) 8.17 d (1H, H³, J 8.1 Hz).

### Example 6

3-methoxybenzanthrone (10) was synthesized according to USSR Patent No. 194828; and Krasovitskii, et al. (B. M. Krasovitskii, et al., Zhurn. Vsesojuz. Khim. Obshchestva [in Russ.], 1967, V. 12, P. 713).

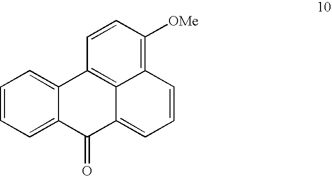

### Example 7

### Synthesis of 3-dimethylaminobenzanthrone (11)

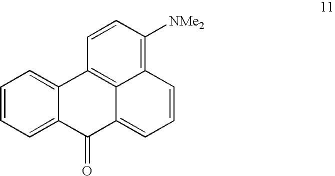

A mixture of 2.4 g (0.01 mol) of 3-aminobenzanthrone and 10 mL (0.1 mol) of dimethylsulphate was heated at 130° C. for 2 h. Then the mixture was diluted with water, neutralized, and the crude product was filtered off, dried, and column purified (benzene, Silica Gel). Yield 1.8 g (67%). M.P. 125-127° C. Red crystals.

### Example 8

### Synthesis of 1,3,3-trimethyl-8-oxo-2,3,4,8-tetrahydro-1H-anthra[1,9-fg]-quinazolin-3-ium hexafluorophosphate (12)

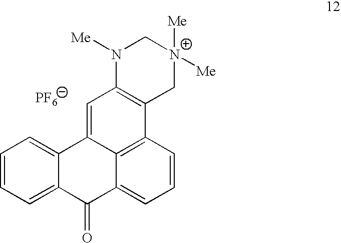

2.73 g (0.1 mol) of 2-dimethylamino benzanthrone was dissolved in 4.6 mL (0.06 mol) of DMF and 3.66 mL (0.04 mol) of POCl₃ were added dropwise at 35-40° C. The mixture was heated with stirring at 80° C. for 1.5 h, cooled down to RT and treated with ice. Then NH₄ PF₆ was added and the obtained precipitate was recrystallized from a water-ethanol (1:1, v/v) mixture. Yield 3.18 g (67%). M.P. 295-297° C. ¹H-NMR, (200 MHz, DMSO-d₆, δ, ppm): 8.79 d (1H, H⁶, J 8.2 Hz), 8.47 d (1H, H⁷, J 7.6 Hz), 8.45 s (1H, H¹), 8.33 d (1H, H¹⁰, J 7.5 Hz), 8.11 d (1H, H⁴, J 8.3 Hz), 7.90 t (2H, H⁸, H⁹, J 7.6 Hz), 7.68 t (1H, H⁵, J 8.0 Hz), 5.15 s (2H, CH₂), 4.86 s (2H, CH₂), 3.51 s (3H, NCH₃), 3.22 s (6H, ⁺N(CH₃)₂). Found, %: C, 55.58; H, 4.62; N, 5.39, C₂₂H₂₁N₂OPF₆. Calculated, %: C, 55.70; H, 4.46; N, 5.91.

### Example 9

### Synthesis of 3-methoxy-7-oxo-7H-benzo[de]anthracene-9-sulfonic acid (13)

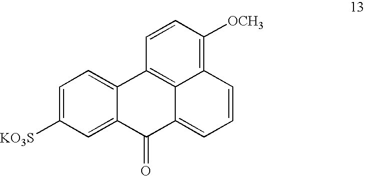

0.5 g (1.92 mmol) of 3-methoxy-7H-benzo[de]anthracen-7-one (10) and 1.5 ml of 9% oleum (fuming sulfuric acid) were mixed and heated at 50° C. with stirring for 8 hours. After cooling reaction mixture was poured into ice and then triturated with concentrated HCl. The obtained red-brown precipitate was filtered off and washed with concentrated HCl. The product was dried in a vacuum desiccator to yield 200 mg (31%) of the product 13.

### Example 10

### Synthesis of 2,2,4-trimethyl-1,2,3,4-tetrahydronaphtho[2,3-f]-quinazolin-2-ium hexafluorophosphate (16)

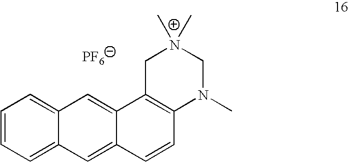

2.21 g (10 mmol) of 2-dimethylaminoanthracene was added at 0-5° C. to a mixture of 4.2 mL (55 mmol) DMF and 1.83 mL (20 mmol) of POCl₃. The mixture was heated with stirring at 80° C. for 3 h, cooled down to RT, treated with ice, neutralized with AcONa, and NH₄ PF₆ was added. The obtained precipitate was recrystallized from aqueous ethanol. Yield 2.74 g (65%). M.P. 255-256° C. ¹H-NMR, (200 MHz, DMSO-d₆, δ, ppm): 8.57 s (1H, H⁹), 8.14 s (1H, H¹⁰), 8.11 d (1H, H⁴, J 9.4 Hz), 8.06 d (2H, H⁵, H⁸, J 8.3 Hz), 7.60 m (2H, H⁶, H⁷, J 8.3 Hz), 7.49 d (1H, H³, J 9.3 Hz), 5.08 s (2H, CH₂), 4.83 s (2H, CH₂), 3.25 s (3H, NCH₃), 3.22 s (6H, ⁺N(CH₃)₂). Found, %: C, 54.22; H, 5.17; N, 6.81, C₁₉H₂₁N₂ PF₆. Calculated, %: C, 54.03; H, 5.01; N, 6.63.

### Example 11

3-Sulfopyrene (18) was synthesized according to Vollmann, et al. (Vollmann, et al, Ann. Chem., 1937, Bd. 531, S. 106).

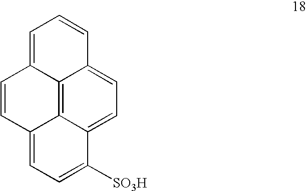

### Example 12

3-Aminopyrene (19a) was synthesized according to Vollman et al. (Vollmann, et al, Ann. Chem., 1937, Bd. 531, S. 109).

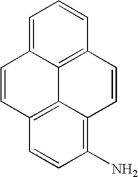

### 19a

### Example 12a

### Synthesis of 6-(6-sulfo-1-pyrenylamino)hexanoic acid (19c)

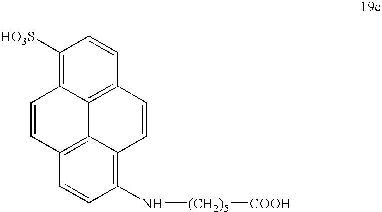

0.2 g (0.63 mmol) of sodium 6-amino-1-pyrenesulfonate was added to a mixture of 0.14 g (0.70 mmol) 6-bromohexanoic acid and 20% solution of sodium hydroxide. The mixture was heated with stirred at 90-95° C. for 2 h, cooled down to RT, neutralized with hydrochloric acid to pH=1, and green fine dust was filtered. The obtained precipitate was twice column purified (Silica gel PR-18, water). Yield 11%. ¹H-NMR (200 MHz, DMSO-d₆, δ, ppm): 8.92 d (1H, arom, J 9.7 Hz), 8.39 d (1H, arom, J 9.7 Hz), 8.32 d (1H, arom, J 7.9 Hz), 8.06 d (1H, arom, J 7.1 Hz), 7.90 d (1H, arom, J 7.9 Hz), 7.89 d (1H, arom, J 8.8 Hz), 7.68 d (1H, arom, J 8.8 Hz), 7.31 d (1H, arom, J 8.3 Hz), 3.48 m (2H, CH₂), 2.25 t (2H, CH₂, J 7.1 Hz), 1.77 t (2H, CH₂, J 7.1 Hz), 1.66-1.37 m (4H, 2CH₂).

### Example 12b

### Synthesis of 7-sulfo-1-pyrenecarboxylic acid (19d)

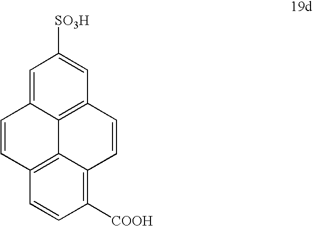

0.72 g (2.93 mmol) of 1-pyrenecarboxylic acid was added to 12.6 g (130 mmol) of sulfuric acid and the mixture was stirred at RT for 3 h, mixed up with ice, neutralized with sodium hydroxide, and obtained yellowish precipitate was twice column purified (Silica gel PR-18, water). Yield 13%. ¹H-NMR (200 MHz, DMSO-d₆, δ, ppm): 9.32 d (1H, arom, J 9.9 Hz), 9.05 d (1H, arom, J 7.9 Hz), 8.46 d (1H, arom, J 8.0 Hz), 8.33 t (1H, arom, J 7.9 Hz), 8.18 (1H, arom, J 7.9 Hz), 8.15 m (1H, arom, J 8.1 Hz), 8.105 s (1H, arom), 8.098 s (1H, arom).

### Example 12c

### Synthesis of 4-oxo-4-(6-sulfo-1-(pyrenyl)butanoic acid (19e)

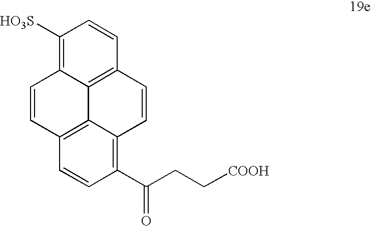

1.2 g (3.64 mmol) of ethyl 4-oxo-4-(1-pyrenyl)butanoate was added to a mixture of 2.54 g (21.82 mmol) of chlorosulfonic acid and 20 mL of chloroform. The mixture was stirred at RT for 5 h. Then the product was extracted with 50 mL of water and hydrolyzed with 0.15 ml of HCl (d=1.19). Green solution was column purified (Silica gel PR-18, water). Yield 13%. ¹H-NMR (200 MHz, DMSO-d₆, δ, ppm): 9.30 d (1H, arom, J 9.5 Hz), 8.72 d (1H, arom, J 9.5 Hz), 8.57 d (1H, arom, J 9.7 Hz), 8.40 d (1H, arom, J 8.2 Hz), 8.30 (1H, arom, J 8.0 Hz), 8.28 m (1H, arom, J 9.4 Hz), 3.49 t (2H, CH₂, J 6.1 Hz), 2.75 t (2H, CH₂, J 6.1 Hz).

### Example 13

### Synthesis of benzoindole derivatives 24 and 25

6-amino-1,3-naphthalenedisulfonic acid disodium salt (22) was purchased from TCI (Product No A0340).

### Synthesis of 6-(1,2-dimethyl-6,8-disulfo-1H-benzo[e]indol-1-yl)hexanoic acid (24)

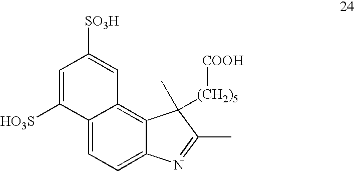

A mixture of 5.0 g (14 mmol) of 6-hydrazino-1,3-naphthalenedisulfonic acid (S. R. Mujumdar, R. B. Mujumdar, C. M. Grant, et al., Bioconjugate Chem., 1996, V. 7, P. 356-362), 2.8 g (15 mmol) of 7-methyl-8-oxononanoic acid, 2.7 g (28 mmol) of potassium acetate and 40 ml of acetic acid was refluxed for 24 hours. The residue was treated with ether, filtered off and washed three times with 20 ml of isopropanol. The product was dried in vacuum desiccator and purified by column chromatography (Li Chroper RP-18, 0.05% trifluoroacetic acid—water) to yield 2.3 g (35%) of the product 24. ¹H-NMR (200 MHz, DMSO-d₆, δ_{H}) 8.90 (1H, d, arom. H), 8.24 (1H, s, arom. H), 8.22 (1H, s, arom. H), 7.69 (1H, d, arom. H), 2.27 (3H, s, 2-CH₃), 2.3-2.1 (2H, m, CH₂), 2.10 (2H, t, CH₂COOH), 1.43 (3H, s, 3-CH₃), 1.35-0.95 (4H, m, (CH₂)₂), 0.6-0.15 (2H, m, —CH₂). UV: λₘₐₓ (abs)=217, 228, 254, 263, 271 nm (methanol).

### Synthesis of tripotassium 6-(1,2-dimethyl-6,8-disulfonato-1H-benzo[e]indol-1-yl)hexanoate

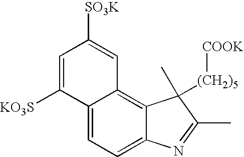

1.25 g (2.7 mmol) of 6-(1,2-dimethyl-6,8-disulfo-1H-benzo[e]indol-1-yl)hexanoic acid (24) were dissolved in 10 ml of methanol and then 450 mg (8 mmol) of potassium hydroxide in 30 ml of isopropanol was added slowly under stirring at RT. The obtained mixture was stirred for 30 minutes at RT. The residue was filtered off, washed with isopropanol, and dried in a vacuum desiccator. Yield 2.26 g (80%). UV: λₘₐₓ(abs)=216, 229, 254, 262.5, 271 nm (water).

### Synthesis of tripotassium 6-[1,2-dimethyl-6,8-disulfonato-3-(3-sulfonatopropyl)-1H-benzo[e]indolium-1-yl]hexanoate (25)

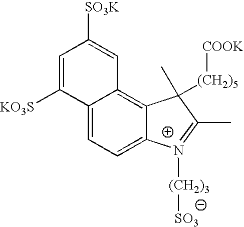

1.26 g (2.2 mmol) of tripotassium 6-(1,2-dimethyl-6,8-disulfonato-1H-benzo[e]indol-1-yl)hexanoate and 1.58 g (13 mmol) of 1,3-propane sultone was melted at 140-150° C. for 12 hours. After cooling the solid formed was treated with acetone. The residue obtained was filtered, washed several times with 10 ml of isopropanol and acetone. The product was dried in a vacuum desiccator. Yield: 1.6 g (99%) of raw product 25. UV: λₘₐₓ (abs)=228 nm, 263 nm, 272 nm, 281 nm (water).

### Example 14

1,3,8,10-tetraoxo-1,3,8,10-tetrahydroisochromeno[6′,5′,4′:10,5,6]anthra-[2,1,9-def]isochromene-5,12-disulfonic acid (27) was synthesized according to Zhubanov, et al. (B. A. Zhubanov, et al. Zhurn. Organ. Khim. [in Russ], 1992, V. 28, P. 1486-1488).

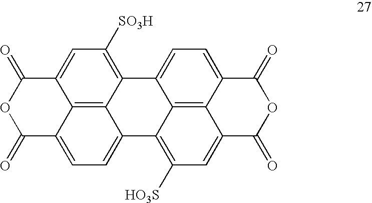

### Example 15

6-amino-3-methyl-2,7-dihydro-3H-naphtho[1,2,3-de]quinoline-2,7-dione (38) was synthesized according to Kazankov (M. V. Kazankov, Zhurn. Vsesojuz. Khim. Obshchestva [in Russ.], 1974, V. 19, P. 64-71).

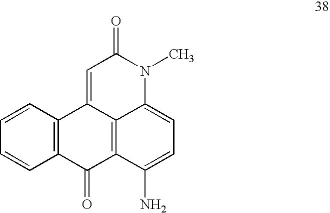

### Example 16

4-dimethylamino-6,11-dihydroanthra[1,2-c][1,2,5]thiadiazole-6,11-dione (39) was synthesized according to (M. V. Gorelik, et al, Khimiya Geterotsykl. Soed. [in Russ.], 1968, No. 3, P. 447-452; M. V. Gorelik, et al, Khimiya Geterotsykl. Soed. [in Russ.], 1971, No. 2, P. 238-243).

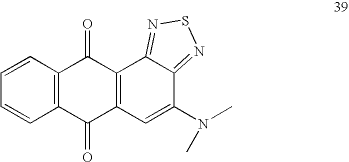

### Example 17

### General Procedure for Labeling of Proteins and Determination of Dye-to-Protein Ratios

Protein labeling reactions were carried out using a 50 mM bicarbonate buffer (pH 9.1). A stock solution of 1 mg of dye in 100 μL of anhydrous DMF was prepared. 10 mg of protein were dissolved in 1 mL of 100 mM bicarbonate buffer (pH 9.1). Dye from the stock solution was added, and the mixture was stirred for 24 h at room temperature.

Unconjugated dye was separated from labelled proteins using gel permeation chromatography with SEPHADEX G50 (0.5 cm×20 cm column) and a 22 mM phosphate buffer solution (pH 7.3) as the eluent. The first colored or/and fluorescent band contained the dye-protein conjugate. A later colored or/and fluorescent band with a much higher retention time contained the separated free dye. A series of labeling reactions as described above were set up to obtain different dye-to-protein ratios. Compared to the free forms, the protein-bound forms of the dyes show distinct changes in their spectral properties.

The dye-to-protein ratio (D/P) gives the number of dye molecules covalently bound to the protein. The D/P ratio was determined according to Mujumdar et al. (R. B. Mujumdar, L. A. Ernst, S. R. Mujumdar, C. J. Lewis, A. S. Waggoner, Bioconjugate Chem., 4 (1993) 105-111). Each dye—BSA conjugate was diluted with phosphate buffer (PB) pH 7.4 to provide the absorbance (A_{conj(λmax)}) in a 5-cm quartz cuvette in the range of 0.15-0.20 at the long-wavelength absorption maximum of the dye—BSA conjugate. For these solutions the absorbances A_{conj(λmax)} at the long-wavelength maximum of the dye and A_{conj(278)} at 278 nm were measured. Then the absorbances of the free dye at 278 nm (A_{dye(278)}) and at the long-wavelength maximum (A_{dye(λmax)}) were taken from the dye absorption spectrum. The dye-to-protein ratio (DIP) were calculated using the following formula:

$$D / P = \frac{{A}_{conj ( λ max )} {ɛ}_{BSA}}{( {A}_{conj ( 278 )} - {xA}_{conj ( λ max )} ) {ɛ}_{dye}} ,$$

where ε_{dye} is the extinction coefficient of the dye at the long-wavelength maximum, and ε_{BSA}=45540 M⁻¹ cm⁻¹ is the extinction coefficient of BSA at 278 nm, and x=A_{dye(278)}/A_{dye(λmax)}.

Covalent Attachment of NHS-Esters to BSA

A stock solution of 1 mg of NHS-ester in 100 μL of anhydrous DMF was prepared. Then 5 mg of BSA was dissolved in 1 mL of a 50 mM bicarbonate buffer, pH 9.0, and a relevant amount of the dye stock solution was added. The mixture was allowed to stir for 3 h at 25° C. Separation of the dye-BSA conjugate from non-conjugated dye was achieved using gel permeation chromatography on a 1.5 cm×25 cm column (stationary phase: SEPHADEX G25; eluent: 67 mM PB, pH 7.4). The fraction with the lowest retention time containing the dye-BSA conjugate was collected.

Covalent Attachment of NHS-Esters to Polyclonal Anti-HSA

385 μL (5.2 mg/mL) of anti-HSA were dissolved in a 750 μL bicarbonate buffer (0.1 M, pH 9.0). 1 mg of NHS-ester is dissolved in 50 μL of DMF and slowly added to the above-prepared protein solution with stirring. After 20 h of stirring, the protein-conjugate was separated from the free dye using SEPHADEX G50 and a phosphate buffer (22 mM, pH 7.2). The first colored or/and fluorescent fraction that is isolated contains the labeled conjugate.

### Example 18

### Synthesis of 5-[4-(2,5-dioxotetrahydro-1H-1-pyrrolyloxycarbonyl)phenyl]-9,9,11-trimethyl-4,6-dioxo-5,6,8,9,10,11-hexahydro-4H-isoquino[4,5-gh]quinazolin-9-ium chloride (3-NHS)

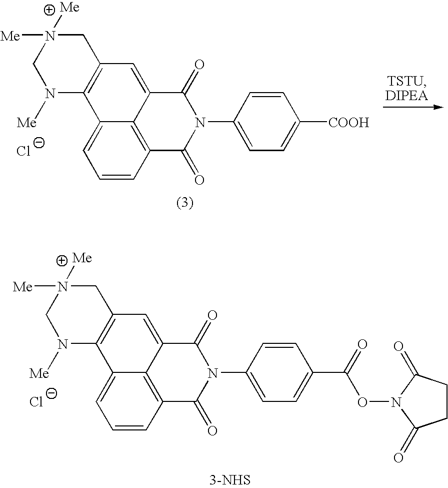

100 mg (0.22 mmol) of 3, 100 mg (0.33 mmol) TSTU, and 76 μL (0.44 mmol) of DIPEA were dissolved in 20 mL of acetonitrile. The obtained solution was stirred at room temperature for 2 h. The reaction was monitored by TLC (RP-18, acetonitrile/water=5/1). After completion, the solvent was removed under reduced pressure and the residue was washed several times with ether, dried and stored in a vacuum desiccator to give NHS ester of 3 with quantitative yield.

### Example 19

### Covalent Attachment of 3-NHS to BSA

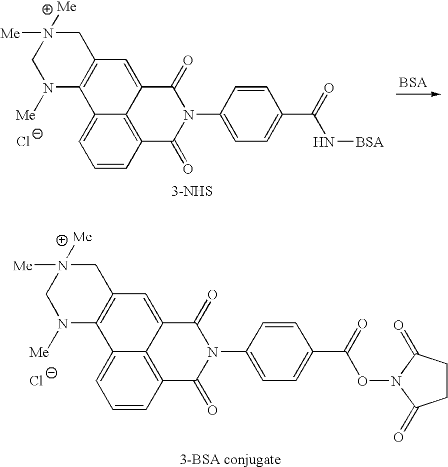

0.8 mg of NHS ester of 3 were dissolved in 80 μL of anhydrous DMF and 17 μL of this solution were added to a solution of 5 mg of BSA in 1 mL of a 50 mM bicarbonate buffer, pH 9.0. The mixture was allowed to stir for 3 h at 25° C. Separation of the dye 3-BSA conjugate from non-conjugated dye was done using a gel permeation chromatography on the 1.5 cm×25 cm column (stationary phase SEPHADEX G25, eluent 67 mM PB of pH 7.4). The fluorescent fraction of yellow color with the lowest retention time containing the dye-BSA conjugate was collected. The obtained D/P ratio was 3.

Using 60 μL of the above dye-NHS stock solution the dye-BSA conjugate with D/P ratio 8 was obtained.

### Example 20

### Synthesis of 6-[2-(2,5-dioxotetrahydro-1H-1-pyrrolyloxycarbonyl)ethyl amino]-1,3-naphthalenedisulfonic acid (23-NHS)

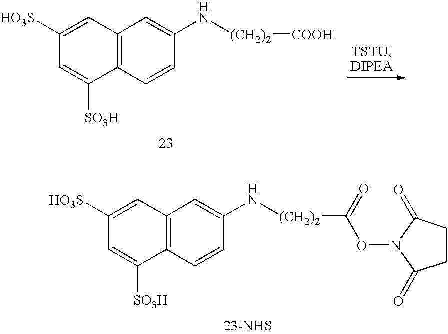

A mixture of 1 mg (2.4 μmol) of 23, 1.1 mg (3.7 μmol) of TSTU, 1 μL (5.7 μmol) of DIPEA, and 100 μL of anhydrous DMF was stirred at room temperature for 2 h. The obtained 23-NHS solution in DMF was used for covalent labeling to protein without additional purification.

### Example 21

### Covalent Attachment of 23-NHS to BSA

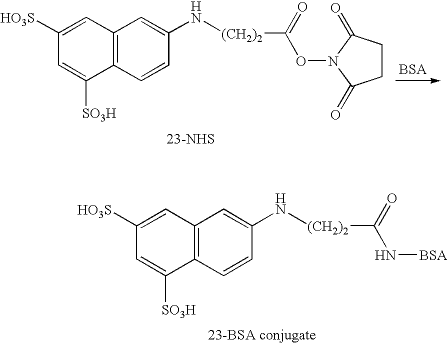

11 mg of BSA were dissolved in 1 mL of a 50 mM of bicarbonate buffer pH 9.0, and 35 μL of the described above 23-NHS solution in DMF were added. The mixture was allowed to stir for 3 h at 25° C. Separation of the dye 23-BSA conjugate from non-conjugated dye was achieved using a gel permeation chromatography on a 1.5 cm×25 cm column (stationary phase SEPHADEX G25, eluent 67 mM PB of pH 7.4). The lowest retention time fluorescent fraction containing the dye-BSA conjugate was collected.

### Example 22

### Synthesis of 3-1-[5-(2,5-dioxotetrahydro-1H-1-pyrrolyloxycarbonyl)pentyl]-1,2-dimethyl-6,8-disulfo-1H-benzo[e]indolium-3-yl-1-propanesulfonate (25-NHS)

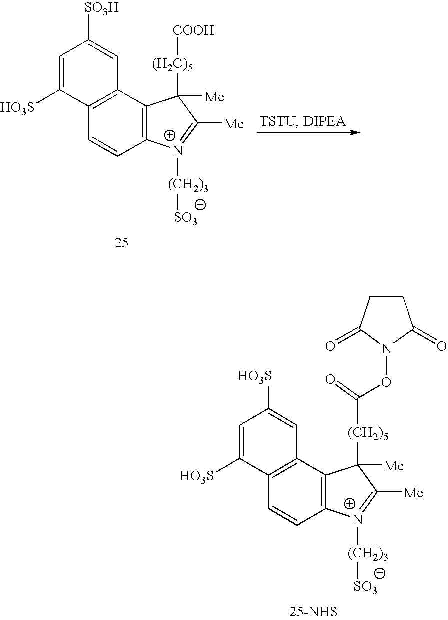

A mixture of 1.2 mg (2.0 μmol) of 25, 1.0 mg (3.3 μmol) of TSTU, 1 μL (5.7 μmol) of DIPEA, and 120 μL of anhydrous DMF was stirred at room temperature for 2 h. The obtained 25-NHS solution in DMF was used for the covalent attachment to protein without additional purification.

### Example 23

### Covalent Attachment of 25-NHS to BSA

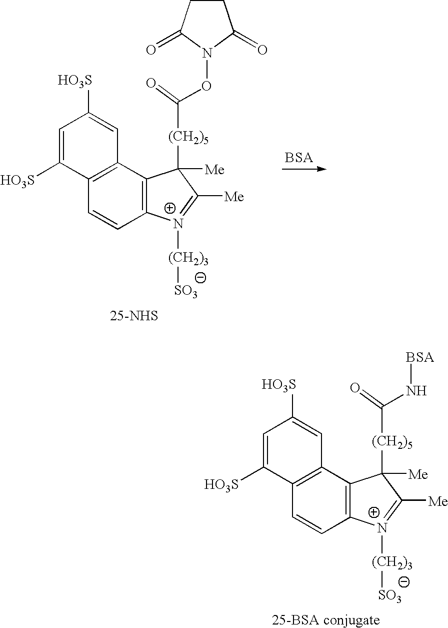

11 mg of BSA were dissolved in 1 mL of a 100 mM of bicarbonate buffer of pH 8.4 and 35 μL of the described above 23-NHS solution in DMF was added. The mixture was allowed to stir for 4 h at 25° C. Separation of the dye 25-BSA conjugate from non-conjugated dye was done using a gel permeation chromatography on the 1.5 cm×25 cm column (stationary phase SEPHADEX G25, eluent 67 mM PB of pH 7.4). The fluorescent fraction with the lowest retention time containing the dye-BSA conjugate was collected.

### Example 24

Reactive functional groups other than NHS have been described in the literature and can be synthesized according to previously described procedures. The syntheses of selective reactive functional groups are described in International Publication no. WO 02/26891 A1.

Spectral Properties of Representative Dyes:

Compounds of the present disclosure invention may have particularly long luminescence lifetimes. For example, selected compounds may exhibit a luminescence lifetime on the order of 4 ns or greater. Such compounds may therefore be particularly useful in lifetime- and polarization-based assays, Fluorescence Lifetime Imaging (FLIM) and other applications where the luminescence lifetime is a parameter of use. In one aspect of the disclosed compounds, they have a luminescence lifetime of 10 ns or longer. In another aspect of the disclosed compounds, they have a luminescence lifetime of between 4 and 30 ns.

The synthesis of selected long-lifetime probes and labels are described in the following Examples. The structures, absorption and emission data as well as the luminescent lifetime in different solvents of specific dyes are given in Table 1.

In one embodiment of the disclosure, the long-lifetime probes and labels are based on naphthalic acid derivatives which have lifetimes in the range of 5 to 23 ns or higher. Representative dyes are listed in Table 1 (compounds 1 to 9) and the synthesis of these dyes is described in the Examples Section (Examples 1 to 5). This class of dyes is perfectly suited for excitation with the blue 404 nm or 436 nm diode lasers and some of these compounds were labeled to BSA to demonstrate that these dyes do maintain long lifetimes in presence of proteins. The data in Table 1 also indicate that the luminescent lifetimes of these compounds is not strongly dependent on the solvent system (see compounds 5 and 6). Compound 6 having a long luminescent lifetime of around 23 ns is a probe and potential label that could have wide-spread use for the development of luminescent assays and sensors for clinical applications and high-throughput screening.

Benzanthrone dyes 10-13 of this invention have longer absorption and emission wavelength (up to 700 nm in water) with lifetimes in the range of 5 to 10 ns in presence of protein.

In another embodiment the lifetime probes and labels are based on anthracene derivatives (Table 1, compounds 14-17). These derivatives have absorption and emission in the blue region of the spectrum with lifetime of 8 ns and higher. In particular reactive derivatives of compound 16 which has a lifetime of 20 ns in water would be very suitable as labels for lifetime based applications.

Pyrenes are known to have long luminescence lifetimes. The sulfo-pyrene compound (Table 1, compound 18) has a lifetime of around 40 ns in water. The sulfonate functional group of this compound can easily be converted into a sulfonyl chloride for covalent labeling to biomolecules. The synthesis is described in Example 11.

Acridine derivatives as shown in Table 1, compounds 20 and 21 have long lifetimes in water which makes them very suitable candidates as labels for lifetime and polarization based assays.

Naphthalene derivatives as shown in Table 1, compounds 22 and 23 have great potential as lifetime probes and labels due to long lifetimes in water and when labeled to proteins. From the data in Table 1 (compounds 23 and 23-BSA) it can be seen that covalent attachment of the naphthalene derivative 23 to proteins does not have a strong effect on the lifetime, which is an important criterion for a label that is used in polarization based assays. Surprisingly the fluorescence lifetime of the disulfo-benzoindole derivative 25 (Table 1) increases more than 3 times from 4.5 ns to 15 ns upon covalent labeling to BSA. This is a very important and unexpected feature and this compound could be used as a lifetime-sensitive tracer in assays and for sensing applications.

Reporter compounds including a 1,3,3-trimethyl-1,2,3,4-tetrahydro pyrimidin-3-ium moiety, where each of the R^{A}, R^{B} and R^{C} substituents are methyl, may demonstrate a particularly enhanced luminescence lifetime. In one embodiment, these compounds may exhibit a luminescence lifetime of greater than 10 ns.

Fused aromatic ring systems are an additional group of lifetime compounds. Some of these derivatives have lifetime in the order of 10 to 20 ns. Importantly the absorption and emission maxima of these compounds are shifted towards longer wavelengths (around 500-600 nm). Reactive versions of these compounds could also be used for labeling and development of luminescence lifetime- and polarization-based assays and sensors.

Table 1. Spectral properties and luminescent lifetimes of representative dyes of this invention

| | | | | | |
|---|---|---|---|---|---|
| Dye | | | λ(abs) | λ(fl) [nm] | Lifetime |
| # | Structure | Solvent | [nm] | (QY [%]) | [ns] |

Naphthalic Acid Derivatives

| | | | | | |
|---|---|---|---|---|---|
| 1 | 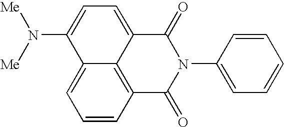 | Toluene | 400 | 493 | 7.5 |
| 2a | 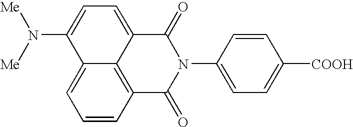 | Water +BSA | 436 | 528 | 4.7 |
| 2b | 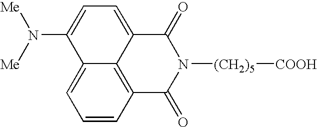 | Water +BSA | 422 | 518 | 9.6 |
| 2c | 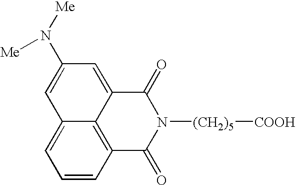 | Ethanol | 458 | 599 (6%) | 6.5 |
| 3 | 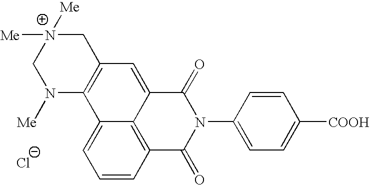 | Water Ethanol | 404 390 | 518 500 | 6.4 8.0 |
| 3-BSA | 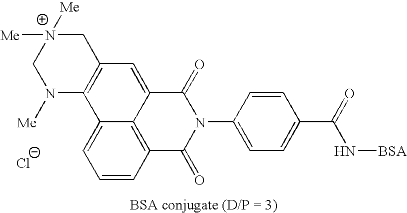 | Water | 406 | 518 | 6.8 |
| 3-BSA | 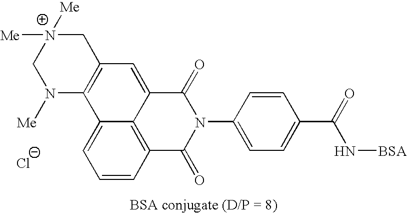 | Water | 406 | 518 | 5.8 |
| 4 | 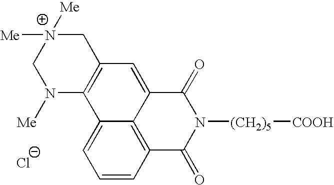 | Water | 402 | 488 | 7.8 |
| 4a | 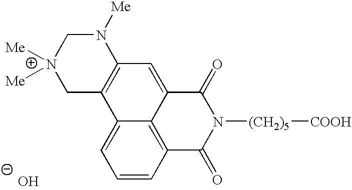 | Water | 425 | 545 (34.5) | 26.1 |
| 5 | 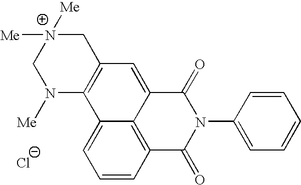 | Water DMF | 420 393 | 520 500 | 9.4 9.0 |
| 6 | 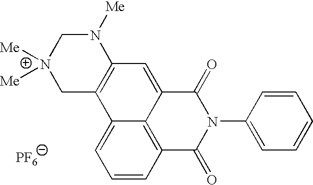 | Ethanol Water | 414 426 | 513 (51%) 545 | 23.3 22.9 |
| 7b | 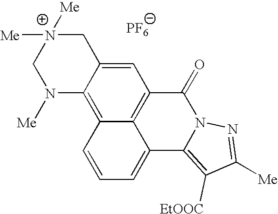 | Ethanol | 421 | 492 (59%) | 5.4 |
| 8 | 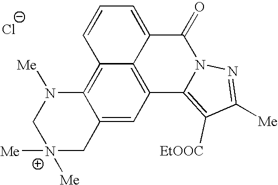 | Ethanol Water Water +BSA | 436 449 449 | 584 (44%) 600 (7%) 600 | 7.8 5.7 6.1 |
| 9 | 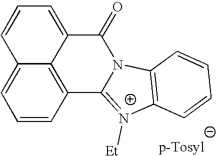 | Water | 390 | 480 | 8.1 |
| 9a | 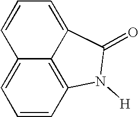 | Ethanol | 361 | 494 (16) | 10.3 |

Benzanthrone Derivatives

| | | | | | |
|---|---|---|---|---|---|
| 10 | 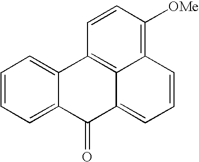 | Ethanol DMF Water +BSA | 435 430 443 | 552 525 542 | 13.6 12.2 9.5 |
| 11 | 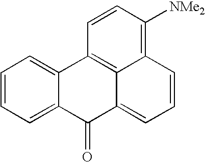 | Ethanol Water Water +BSA | 470 458 465 | 666 (11%) 696 637 | 4.0 1.5 6.5 |
| 12 | 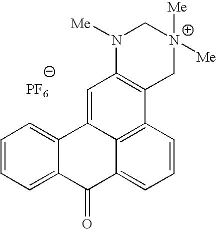 | Ethanol Water Water +BSA | 439 450 440 | 593 (22%) 645 (2%) 594 | 17.0 8.1 5.2 |
| 13 | 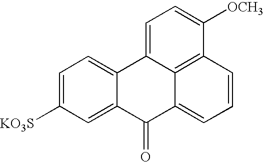 | Water | 447 | 575 | 9.4 |

Anthracene Derivatives

| | | | | | |
|---|---|---|---|---|---|
| 14 | 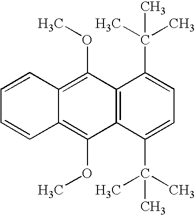 | Toluene | 380 | 445 | 8.3 |
| 15 | 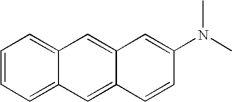 | Ethanol | 416 | 506 (26%) | 11.9 |
| 16 | 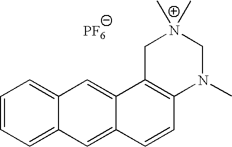 | Water | 398 | 481 (40%) | 20.9 |
| 17 | 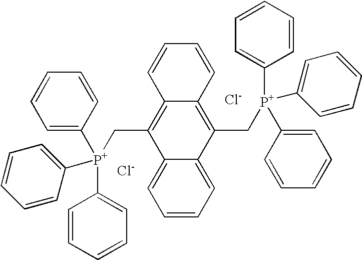 | Water DMF Ethanol | 412 390 412 | 447 450 447 | 8.3 7.9 7.7 |

Pyrene Derivatives

| | | | | | |
|---|---|---|---|---|---|
| 18 | 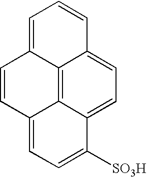 | Water | 346 | 376 | 39.5 |
| 19a | 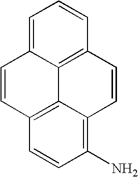 | Ethanol | 359 | 431 | 4.1 |
| 19b | 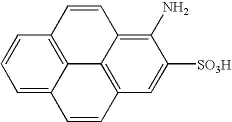 | Water | 364 | 474 | 4.9 |
| 19c | 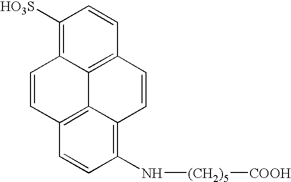 | Water Water +BSA | 410, 376, 432 | 492 (45%) | 5.3 5.2 |
| 19c- BSA | 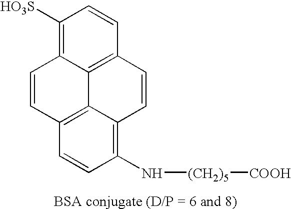 | Water | 432 | 479 (8%) | 5.4 |
| 19d | 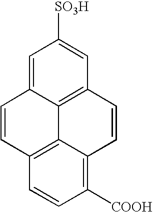 | Water Water +BSA | 358, 352 | 398 (55%) | 14.4 9.8 |
| 19d- BSA | 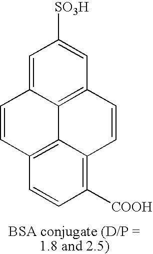 | Water monomer eximer | | 386, 397 485 | 7.3 37.0 |
| 19e | 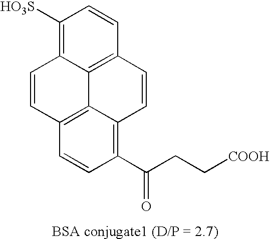 | Water | 357, 280 | 419, 391, 377 | 14.4 |
| 19e | 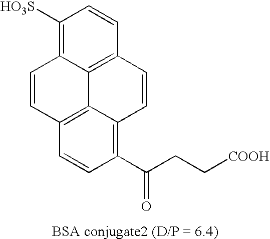 | Water monomer eximer | (358, 282) | (526, 443, 418, 395, 376) | 16.0 9.8 |

Acridine Derivatives

| | | | | | |
|---|---|---|---|---|---|
| 20 | 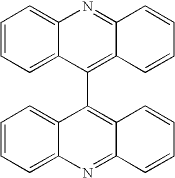 | Ethanol | 398 | 412, 435 | 9.6 |
| 21 | 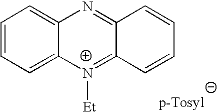 | Water | 354 | 448 | 14.8 |

Naphthalene Derivatives

| | | | | | |
|---|---|---|---|---|---|
| 22 | 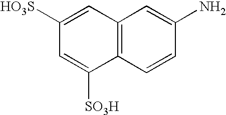 | Water | 350 | 472 | 22.5 |
| 23 | 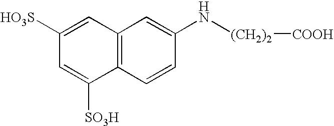 | Water | 380 | 480 | 21.5 |
| 23-BSA | 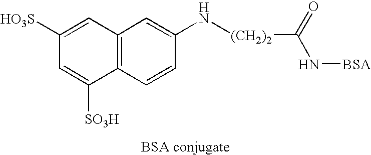 | Water | 383 | 469 | 19.6 |
| 24 | 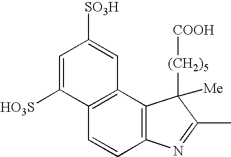 | Water | 348 | 382 | 5.5 |
| 24-BSA | 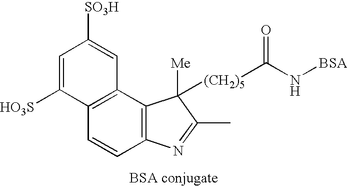 | Water | 348 | 376 | 4.3 |
| 25 | 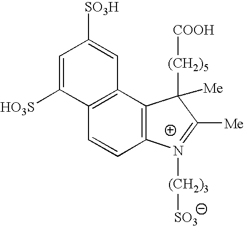 | Water | 345 | 385 | 4.5 |
| 25-BSA | 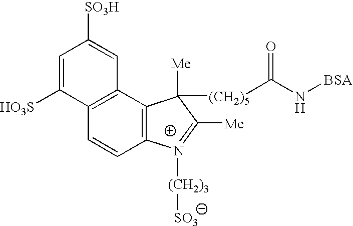 | Water | 330 | 357, 495 | 15.2 |
| 26 | 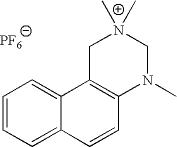 | Ethanol | 341 | 384 | 5.7 |

Perylene-tetracarboxylic
Acid Derivatives

| | | | | | |
|---|---|---|---|---|---|
| 27 | 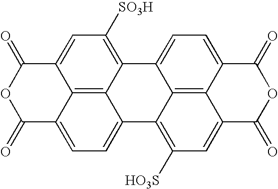 | Water | 503 | 532 | 7.1 |

Other Fused Systems

| | | | | | |
|---|---|---|---|---|---|
| 28 | 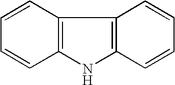 | Ethanol | 337 | 344, 359 | 6.6 |
| 29 | 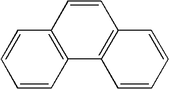 | Ethanol | 376 | 400 | 4.1 |
| 30 | 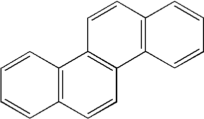 | Ethanol | 361 | 381 | 16.1 |
| 31 | 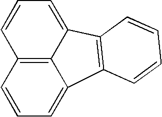 | Ethanol | 358 | 460 | 28.3 |
| 32 | 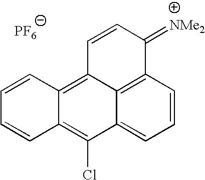 | Ethanol | 571 | 668 (5%) | 3.9 |
| 33 | 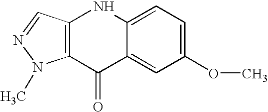 | Ethanol DMF | 397 395 | 434 425 | 10.1 9.3 |
| 34 | 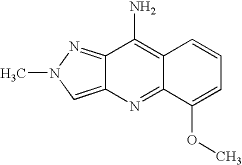 | Ethanol DMF | 425 425 | 476 457 | 14.2 11.2 |
| 35 | 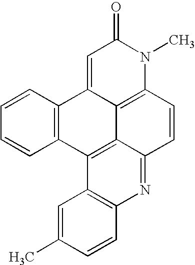 | Toluene Methanol | 496 476 | 517 528 | 12.2 9.7 |
| 36 | 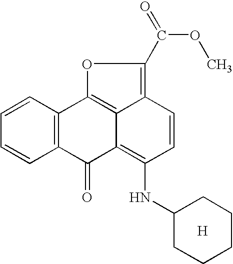 | Toluene | 516 | 571, 609 | 10.9 |
| 37 | 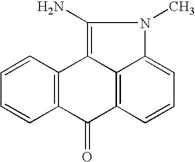 | Toluene Ethanol | 451 485 | 472 505 (75%) | 6.5 10.1 |
| 38 | 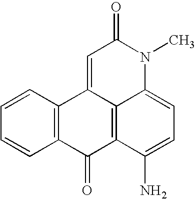 | Ethanol | 510 | 570 | 9.3 |
| 39 | 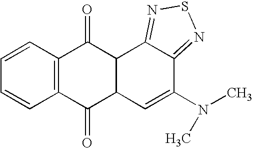 | Toluene | 535 | 610 | 13.5 |

Description of Applications of the Invention

The reporter compounds disclosed above exhibit utility for any assay that utilizes colorimetric or luminescent labeling. In general, a variety of useful assay formats exist that may be improved by the use of the presently disclosed compounds.

For example, the assay may be a competitive assay that includes a recognition moiety, a binding partner, and an analyte. Binding partners and analytes may be selected from the group consisting of biomolecules, drugs, and polymers, among others. In some competitive assay formats, one or more components are labeled with photoluminescent compounds in accordance with the invention. For example, the binding partner may be labeled with such a photoluminescent compound, and the displacement of the compound from an immobilized recognition moiety may be detected by the appearance of fluorescence in a liquid phase of the assay. In other competitive assay formats, an immobilized enzyme may be used to form a complex with the fluorophore-conjugated substrate.

Some of the present reporter molecules include specific moieties for specific labeling of protein tyrosine phosphatases, as well as other phosphatases as described by Zhu, Q., et al. (Tetrahedron Letters, 44, 2669 (2003).

The binding of antagonists to a receptor can be assayed by a competitive binding method in so-called ligand/receptor assays. In such assays, a labeled antagonist competes with an unlabeled ligand for the receptor binding site. One of the binding partners can be, but not necessarily has to be, immobilized. Such assays may also be performed in microplates. Immobilization can be achieved via covalent attachment to the well wall or to the surface of beads.

Other preferred assay formats are immunological assays. There are several such assay formats, including competitive binding assays, in which labeled and unlabeled antigens compete for the binding sites on the surface of an antibody (binding material). Typically, there are incubation times required to provide sufficient time for equilibration. Such assays can be performed in a heterogeneous or homogeneous fashion.

Sandwich assays may use secondary antibodies and excess binding material may be removed from the analyte by a washing step.

Other types of reactions include binding between avidin and biotin, protein A and immunoglobulins, lectins and sugars (e.g., concanavalin A and glucose).

Certain dyes of the invention are charged due to the presence sulfonic or a quarternary nitrogen atoms in a ring structure (see compounds 3-9, 12, 16 in Table 1). These compounds are typically impermeant to membranes of biological cells. In this case treatments such as electroporation and shock osmosis can be used to introduce the dye into the cell. Alternatively, such dyes can be physically inserted into the cells by pressure microinjection, scrape loading etc.

The reporter compounds described here also may be used in sequencing nucleic acids and peptides. For example, fluorescently-labeled oligonucleotides may be used to trace DNA fragments. Other applications of labeled DNA primers include fluorescence in-situ hybridization methods (FISH) and for single nucleotide polymorphism (SNIPS) applications, among others.

Multicolor labeling experiments may permit different biochemical parameters to be monitored simultaneously. For this purpose, two or more reporter compounds are introduced into the biological system to report on different biochemical functions. The technique can be applied to fluorescence in-situ hybridization (FISH), DNA sequencing, fluorescence microscopy, and flow cytometry among others. One way to achieve multicolor analysis is to label biomolecules such as nucleotides, proteins or DNA primers with different luminescent reporters having distinct luminescence properties (e.g. excitation or emission maxima). Multi-lifetime analysis on the other hand is based on labeling with reporters that have the same excitation and emission maxima but differ due to their distinct luminescence lifetimes. Compounds of this invention have lifetimes in the range from 4 ns to 40 ns and higher and can therefore be easily differentiated by measuring the luminescence lifetime or a relevant parameter (e.g. phase angle).

Phosphoramidites are useful functionalities for the covalent attachment of dyes to oligos in automated oligonucleotide synthesizers. They are easily obtained by reacting the hydroxyalkyl-modified dyes of the invention with 2-cyanoethyl-tetraisopropyl-phosphorodiamidite and 1-H tetrazole in methylene chloride.

The simultaneous use of FISH (fluorescence in-situ hybridization) probes in combination with different fluorophores is useful for the detection of chromosomal translocations, for gene mapping on chromosomes, and for tumor diagnosis, to name only a few applications. One way to achieve simultaneous detection of multiple sequences is to use combinatorial labeling. The second way is to label each nucleic acid probe with a luminophore with distinct properties (e.g lifetime). Conjugates can be synthesized from this invention and can be used in a multicolor-multilifetime multisequence analysis approach.

In another approach the dyes of the invention might be used to directly stain or label a sample so that the sample can be identified and or quantitated. Such dyes might be added/labeled to a target analyte as a tracer. Such tracers could be used e.g. in photodynamic therapy where the labeled compound is irradiated with a light source and thus producing singlet oxygen that helps to destroy tumor cells and diseased tissue samples.

The reporter compounds of the invention can also be used in screening assays for a combinatorial library of compounds. The compounds can be screened for a number of characteristics, including their specificity and avidity for a particular recognition moiety.

Assays for screening a library of compounds are well known. A screening assay is used to determine compounds that bind to a target molecule, and thereby create a signal change which is generated by a labeled ligand bound to the target molecule. Such assays allow screening of compounds that act as agonists or antagonists of a receptor, or that disrupt a protein-protein interaction. It also can be used to detect hybridization or binding of DNA and/or RNA.

Other screening assays are based on compounds that affect the enzyme activity. For such purposes, quenched enzyme substrates of the invention could be used to trace the interaction with the substrate. In this approach, the cleavage of the fluorescent substrate leads to a change in the spectral properties such as the excitation and emission maxima, intensity, polarization and/or lifetime, which allows to distinguish between the free and the bound luminophore.

The dye compounds are also useful for use as biological stains. There may be limitations in some instances to the use of the above compounds as labels. For example, typically only a limited number of dyes may be attached to a biomolecules without altering the fluorescence properties of the dyes (e.g. quantum yields, lifetime, emission characteristics, etc.) and/or the biological activity of the bioconjugate. Typically quantum yields may be reduced at higher degrees of labeling. Encapsulation into beads offers a means to overcome the above limitation for the use of such compounds as fluorescent markers. Fluorescent beads and polymeric materials are becoming increasingly attractive as labels and materials for bioanalytical and sensing applications. Various companies offer particles with defined sizes ranging from nanometers to micrometers. Noncovalent encapsulation in beads may be achieved by swelling the polymer in an organic solvent, such as toluene or chloroform, containing the dye. Covalent encapsulation may be achieved using appropriate reactive functional groups on both the polymer and the dyes.

In general, hydrophobic versions of the invention may be used for non-covalent encapsulation in polymers, and one or more dyes could be introduced at the same time. Surface-reactive fluorescent particles allow covalent attachment to molecules of biological interest, such as antigens, antibodies, receptors etc. Hydrophobic versions of the invention such as dye having lipophilic substituents such as phospholipids will non-covalently associate with lipids, liposomes, lipoproteins. They are also useful for probing membrane structure and membrane potentials.

Compounds of this invention may also be attached to the surface of metallic nanoparticles such as gold or silver nanoparticles or metal-coated surfaces. It has recently been demonstrated that fluorescent molecules may show increased quantum yields near metallic nanostructures e.g. gold or silver nanoparticles (O. Kulakovich et al. Nanoletters 2 (12) 1449-52, 2002). This enhanced fluorescence may be attributable to the presence of a locally enhanced electromagnetic field around metal nanostructures. The changes in the photophysical properties of a fluorophore in the vicinity of the metal surface may be used to develop novel assays and sensors. In one example the nanoparticle may be labeled with one member of a specific binding pair (antibody, protein, receptor etc) and the complementary member (antigen, ligand) may be labeled with a fluorescent molecule in such a way that the interaction of both binding partners leads to an detectable change in one or more fluorescence properties (such as intensity, polarization, quantum yield, lifetime, phase angle among others). Replacement of the labeled binding partner from the metal surface may lead to a change in fluorescence, which can then be used to detect and/or quantify an analyte.

Conventional fluorophores have lifetimes in the range of 100 ps to 4 ns. It is known that the luminescence lifetime of a fluorophore near a metallic nanostructure exhibits shorter lifetimes thus the lifetime of conventional labels will be shortened to an extend that measurement with inexpensive instrumentation is not possible. Dyes of this invention have in average 10 times longer lifetimes than conventional dyes and will therefore allow the use of inexpensive instrumentation even in presence of metallic nanostructures.

Gold colloids can be synthesized by citrate reduction of a diluted aqueous HAuCl₄ solution. These gold nanoparticles are negatively charged due to chemisorption of citrate ions. Surface functionalization may be achieved by reacting the nanoparticles with thiolated linker groups containing amino or carboxy functions. In another approach, thiolated biomolecules are used directly for coupling to these particles.

Analytes

The invention may be used to detect an analyte that interacts with a recognition moiety in a detectable manner. As such, the invention can be attached to a recognition moiety which is known to those of skill in the art. Such recognition moieties allow the detection of specific analytes. Examples are pH-, or potassium sensing molecules, e.g., synthesized by introduction of potassium chelators such as crown-ethers (aza crowns, thia crowns etc). Dyes with N—H substitution in the heterocyclic rings are known to exhibit pH-sensitive absorption and emission (S. Miltsov et al., Tetrahedron Lett. 40: 4067-68, (1999), M. E. Cooper et al., J. Chem. Soc. Chem. Commun. 2000, 2323-2324), Calcium-sensors based on the BAPTA (1,2-Bis(2-aminophenoxy)ethan-N,N,N′,N′-tetra-aceticacic) chelating moiety are frequently used to trace intracellular ion concentrations. The combination of a compound of the invention and the calcium-binding moiety BAPTA may lead to new long-wavelength absorbing and emitting Ca-sensors which could be used for determination of intra- and extracellular calcium concentrations (Akkaya et al. Tetrahedron Lett. 38:4513-4516 (1997). Additionally, or in the alternative, reporter compounds already having a plurality of carboxyl functional groups may be directly used for sensing and/or quantifying physiologically and environmentally relevant ions.

Fluorescence Methods

Dyes of this disclosure may be useful in particular because of their long luminescent lifetimes up to 40 ns and higher. The long nanosecond lifetime of the dyes and dye-protein conjugates may allow the use of relatively inexpensive instrumentation that employs laser diodes for excitation. Typical assays based on the measurement of the fluorescence lifetime as a parameter include for example FRET (fluorescence resonance energy transfer) assays. The binding between a fluorescent donor labeled species (typically an antigen, or a ligand) and a fluorescent acceptor labeled species may be accompanied by a change in the intensity and/or the fluorescence lifetime. The lifetime can be measured using intensity-based (Time-correlated single photon counting TCSPC) or phase-modulation-based methods (J. R. LAKOWICZ, PRINCIPLES OF FLUORESCENCE SPECTROSCOPY (2^{nd} Ed. 1999)). Due to the broad range of lifetimes exhibited by these dyes they can be used simultaneously in multi-lifetime multi-analyte assays (see above).

Dyes of this disclosure may also exhibit high intrinsic polarization in the absence of rotational motion, making them useful as tracers in fluorescence polarization (FP) assays. Fluorescence polarization immunoassays (FPI) are widely applied to quantify low molecular weight antigens. The assays are based on polarization measurements of antigens labeled with fluorescent probes. The requirement for polarization probes used in FPIs is that emission from the unbound labeled antigen be depolarized and increase upon binding to the antibody. Low molecular weight species labeled with the compounds of the invention can be used in such binding assays, and the unknown analyte concentration can be determined by the change in polarized emission from the fluorescent tracer molecule. The longer luminescent lifetimes of the present labels permit the measurement of higher molecular weight antigens in a fluorescence polarization assay because the MW of the labeled analyte that can be measured in such a polarization assay is directly dependent on the luminescence lifetime of the label (E. Terpetschnig et al. Biophys J. 68(1):342-50, 1995).

Compositions and Kits

The invention also provides compositions, kits and integrated systems for practicing the various aspects and embodiments of the invention, including producing the novel compounds and practicing of assays. Such kits and systems may include a reporter compound as described above, and may optionally include one or more of solvents, buffers, calibration standards, enzymes, enzyme substrates, and additional reporter compounds having similar or distinctly different optical properties.

Although the invention has been disclosed in preferred forms, the specific embodiments thereof as disclosed and illustrated herein are not to be considered in a limiting sense, because numerous variations are possible. Applicant regards the subject matter of his invention to include all novel and nonobvious combinations and subcombinations of the various elements, features, functions, and/or properties disclosed herein. No single element, feature, function, or property of the disclosed embodiments is essential. The following claims define certain combinations and subcombinations of elements, features, functions, and/or properties that are regarded as novel and nonobvious. Other combinations and subcombinations may be claimed through amendment of the present claims or presentation of new claims in this or a related application. Such claims, whether they are broader, narrower, or equal in scope to the original claims, also are regarded as included within the subject matter of applicant's invention.

We claim:

1. A composition of matter comprising a luminescent reporter compound according to the formula: 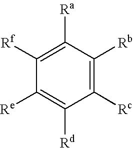
wherein
each R^{a}—R^{f} is independently selected from the group consisting of H, alkyl, alkoxy, amino, alkylamino, dialkylamino, alkenyl, alkynyl, aryl, halogen, sulfo, carboxy, formyl, acetyl, formylmethyl, sulfate, phosphate, phosphonate, ammonium, alkylammonium, cyano, nitro, azido, heterocyclic, substituted heterocyclic, reactive aliphatic and reactive aromatic groups; and
wherein at least one pair of adjacent substituents (R^{a}, R^{b}), (R^{b}, R^{c}), (R^{c}, R^{d}), (R^{d}, R^{e}), (R^{e}, R^{f}), (R^{f}, R^{a}) is a substituted cyclic or polycyclic group W¹, W², W³, W⁴, W⁵, W⁶, W⁷, W⁸, W⁹; or
at least one set of three substituents (R^{a}, R^{b}, R^{c}), (R^{b}, R^{c}, R^{d}), (R^{c}, R^{d}, R^{e}), (R^{d}, R^{e}, R^{f}), (R^{e}, R^{f}, R^{a}) is a substituted cyclic or polycyclic group W⁹, W¹⁰, W¹¹, W¹²; 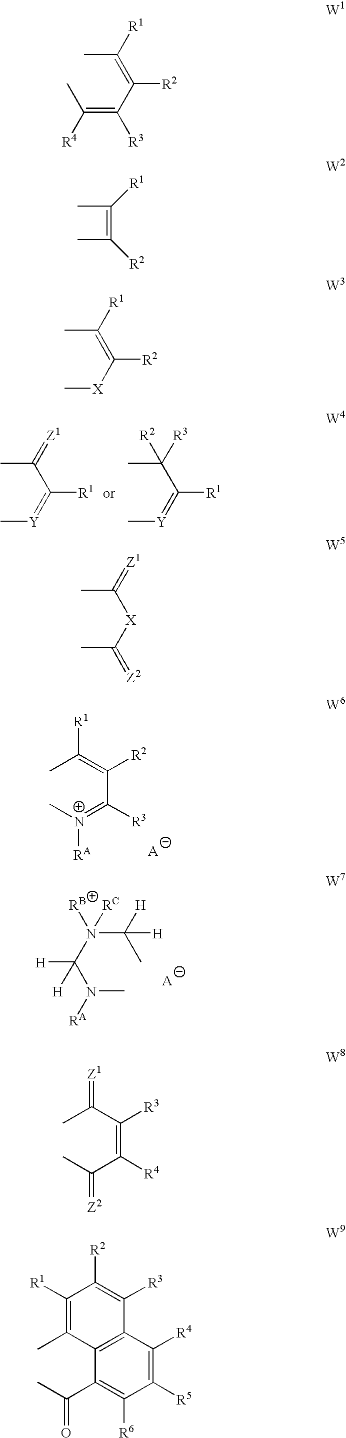 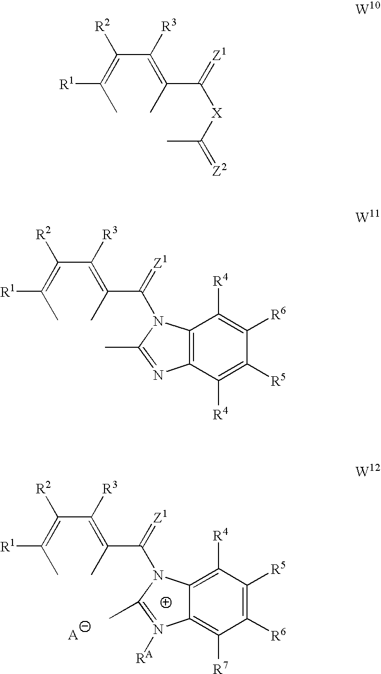
wherein
each R¹-R⁷ is independently selected from the group consisting of H, alkyl, alkoxy, amino, alkylamino, dialkylamino, alkenyl, alkynyl, aryl, halogen, sulfo, carboxy, formyl, acetyl, formylmethyl, sulfate, phosphate, phosphonate, ammonium, alkylammonium, cyano, nitro, azido, heterocyclic, substituted heterocyclic, reactive aliphatic and reactive aromatic groups
X is selected from the group consisting of C(R^{B})(R^{C}), O, S, Se, N—R^{A};
Y is selected from the group consisting of CR^{A}, N, ⁺N—R^{A}, O⁺, S⁺;
Z¹, Z² are independently selected from the group consisting of ═O, ═S, ═Se, ═Te, ═N—R^{A}, and ═C(R^{B})(R^{C})
R^{A} is selected from H, aliphatic groups, alicyclic groups, alkylaryl groups, aromatic groups, -L-S_{c}, -L-R^{x}, -L-R^{±} among others.
R^{B}, R^{C} are independently selected from H, aliphatic groups, alicyclic groups, alkylaryl groups, aromatic groups, -L-S_{c}, -L-R^{x}, -L-R^{±} among others, or adjacent R^{B}, R^{C} form a cyclic group.
L is a covalent linkage that is linear or branched, cyclic or heterocyclic, saturated or unsaturated, having 1-20 nonhydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen or nitrogen-platinum bonds, or aromatic or heteroaromatic bonds;
R^{x} is a reactive group;
S_{c} is a conjugated substance;
R^{±} is an ionic group;
A⁻ is any anion;
provided that the compound is luminescent and has a luminescence lifetime on the order of 4 nanoseconds (ns) or longer.

2. The composition of claim 1, where the compound has the formula:

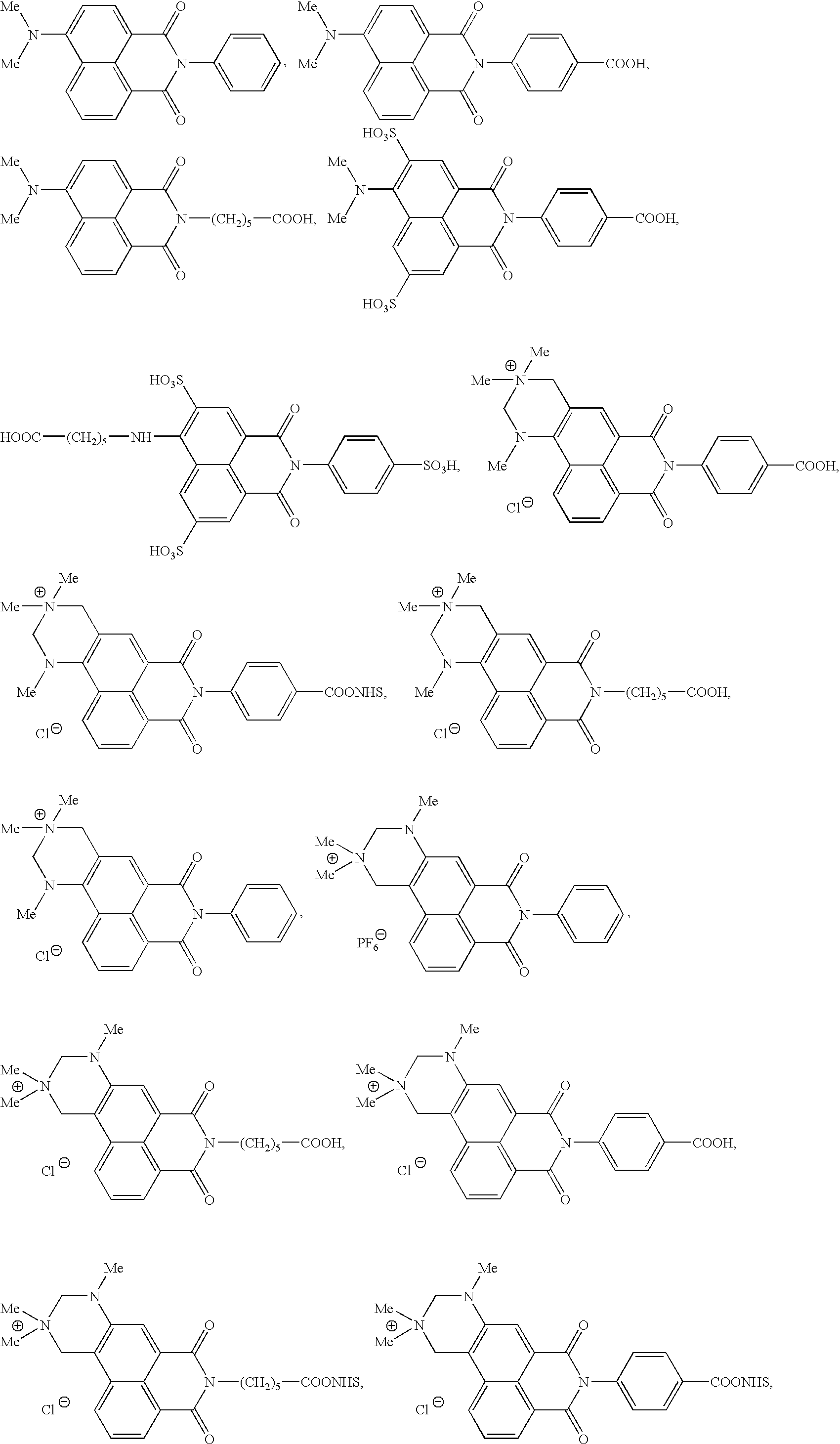

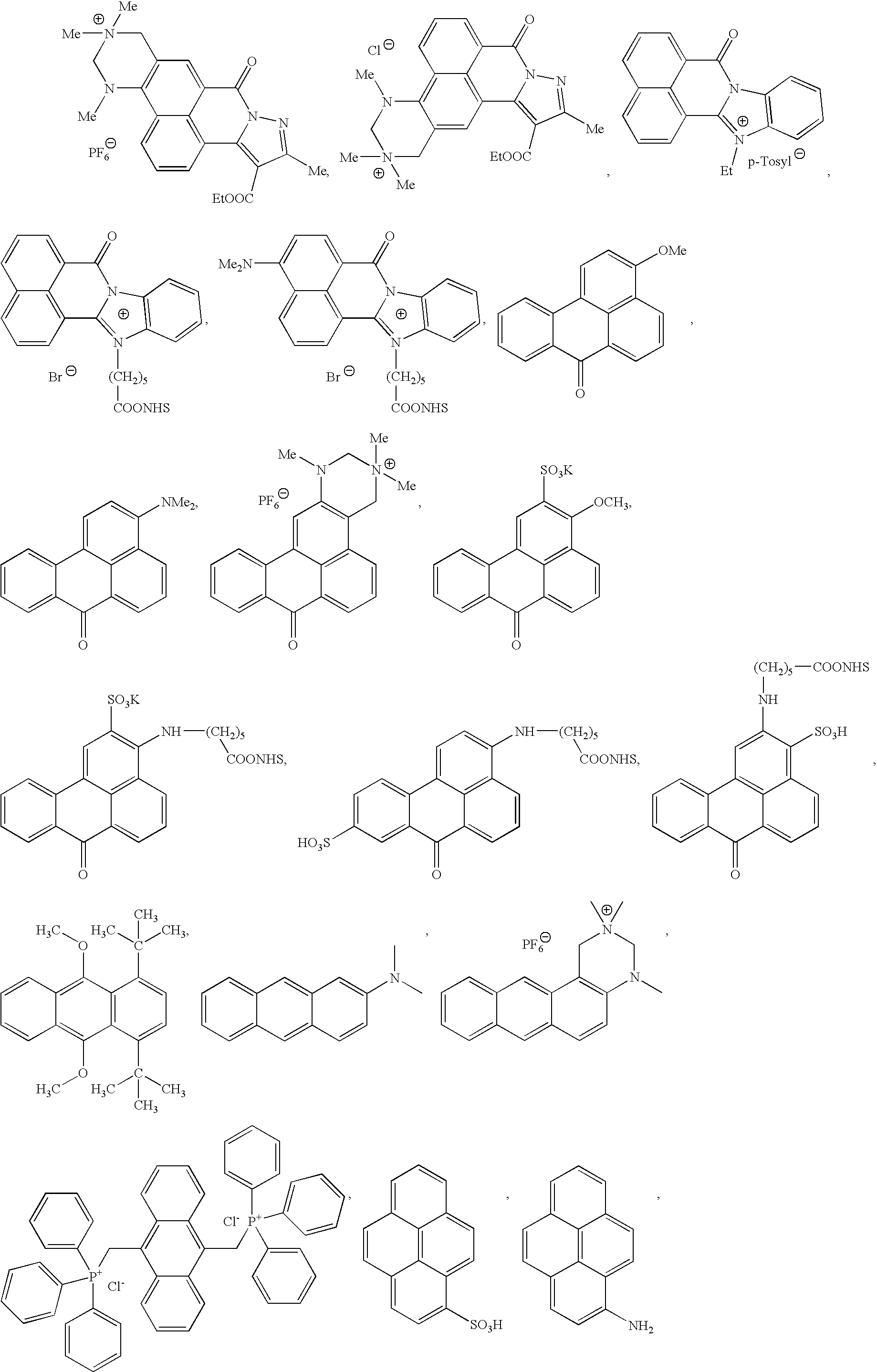

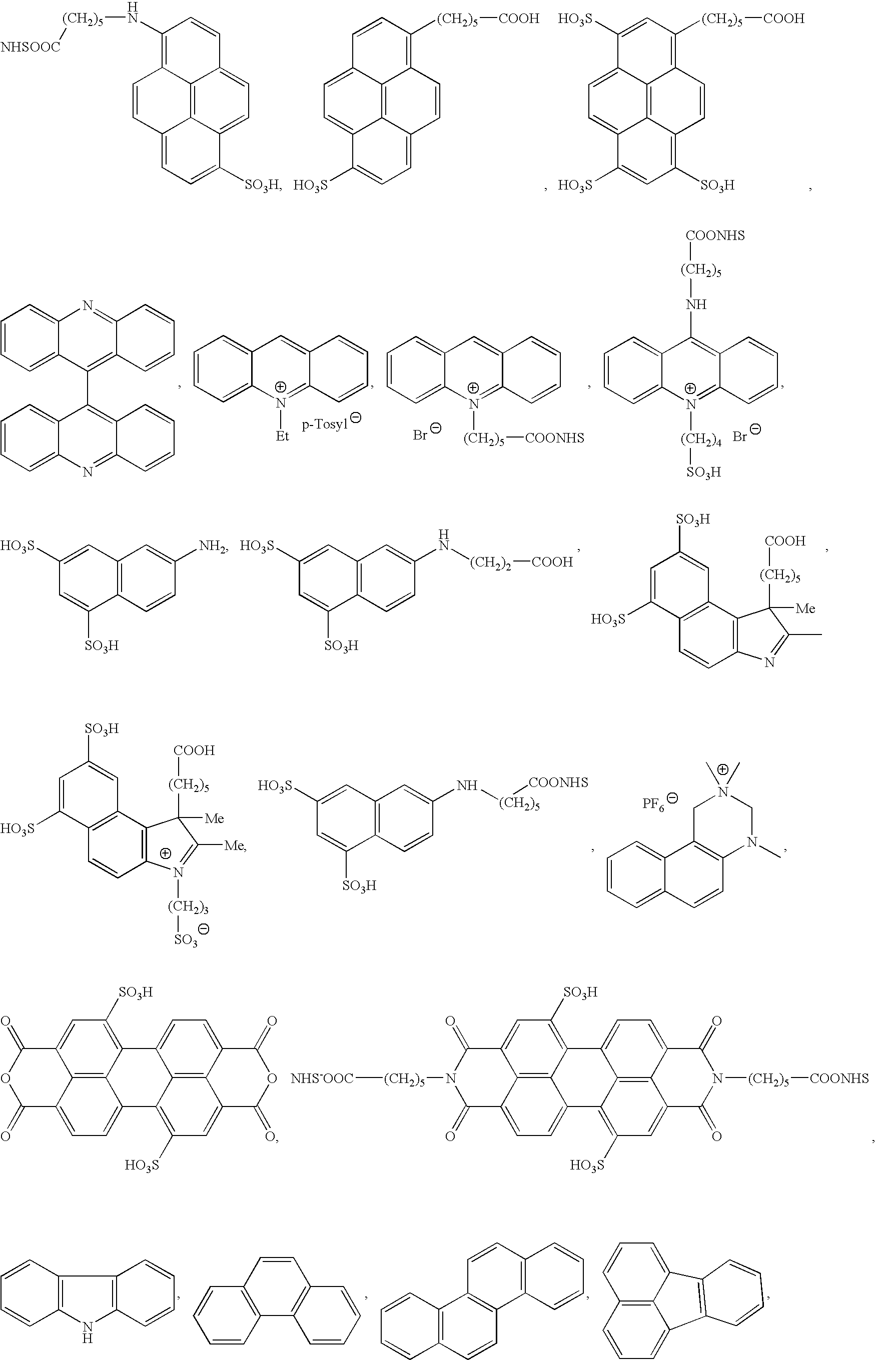

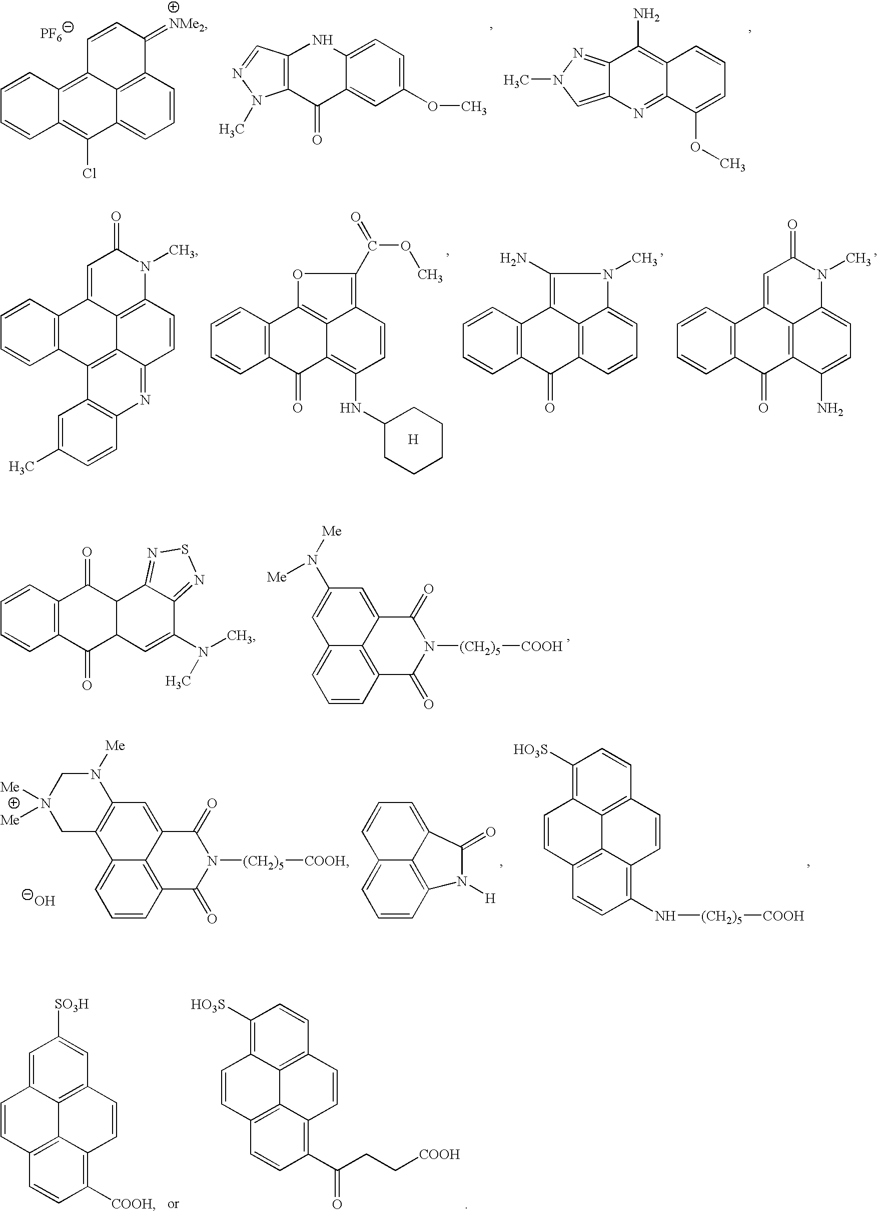

3. The composition of claim 1, wherein at least one substituent includes a reactive group R^{x}.

4. The composition of claim 3, wherein the reactive group R^{x} is selected to cross-react with amine moieties from the group consisting of N-hydroxysuccinimide esters, isothiocyanates, sulfonylhalogenides, and anhydrides.

5. The composition of claim 3, wherein the reactive group R^{x} is selected to cross-react with thiol moieties from the group consisting of iodoacetamides and maleimides.

6. The composition of claim 3, wherein the reactive group R^{x} is selected to cross-react with nucleic acids from the group consisting of phosphoramidites.

7. The composition of claim 1, wherein at least one substituent includes a linked carrier L-S_{c}.

8. The composition of claim 7, wherein the carrier S_{c} is selected from the group consisting of proteins, DNA, polypeptides, polynucleotides, beads, microplate well surfaces, lipids, small-molecule drugs, lectins, pharmacological agents and metallic nanoparticles.

9. The composition of claim 7, wherein the carrier S_{c} is a polypeptide or a polynucleotide.

10. The composition of claim 1, further comprising a carrier S_{c}, which is associated covalently with the reporter compound through reaction with a reactive group on at least one substituent.

11. The composition of claim 1, wherein at least one substituent is an R^{±} capable of increasing the hydrophilicity of the entire compound.

12. The composition of claim 11, wherein the R^{±} substituent is selected from the group consisting of —CH₂—CONH—SO₂-Me, SO₃—, COO—, pO₃²⁻, O—PO₃²⁻, PO₃R⁻, O—PO₃R⁻ and N(R)₃⁺, wherein each R is independently an aliphatic or aromatic moiety.

13. The composition of claim 1, wherein the compound substituents are selected so that the compound is electrically neutral.

14. The composition of claim 1, wherein the compound substituents are selected so that the reporter compound contains a maximal positive or negative net charge, maximizing its solubility in aqueous media and reducing its aggregation tendency in water and when covalently bound to proteins or other biomolecules.

15. The composition of claim 1, wherein the reporter compound is capable of covalently reacting with at least one of biological cells, DNA, lipids, nucleotides, polymers, proteins, lectins, pharmacological agents and solid surfaces.

16. The composition of claim 1, wherein the reporter compound is covalently or noncovalently associated with at least one of biological cells, DNA, oligonucleotides, lipids, nucleotides, polymers, peptides, proteins, and pharmacological agents.

17. The composition of claim 1, further comprising a second reporter compound selected from the group consisting of luminophores and chromophores.

18. The composition of claim 1, wherein one of the first or second reporter compounds is an energy transfer donor and the other reporter compound is an energy transfer acceptor.

19. The composition of claim 1, wherein the reporter compound is used in a luminescence lifetime-based application.

20. The composition of claim 1, wherein the reporter compound is used in a luminescence polarization-based application.

21. A composition of claim 1 having the formula 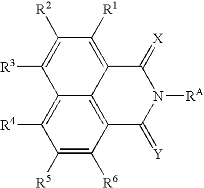
where X is independently selected from O, S and N—R^{A}; R¹-R⁶ are independently H, aliphatic groups, aromatic groups, halogen, amino, nitro, sulfo, or substituted amino groups; R² and R³ or R⁴ and R⁵ are independently selected from W⁷, with R^{A}, R^{a}, R^{b}, and R^{c} independently being H, alkyl, aryl, -L-S_{c}, -L-R^{x}, and -L-R^{±}; and where X═C—N—R^{a} may be a part of a substituted heterocyclic or condensed heterocyclic ring structure.

22. The composition of claim 21, wherein X═C—N—R^{A} is part of a substituted pyrazole or benzimidazolium system.

23. The composition of claim 21, wherein X═O; R¹, R⁴, R⁵ and R⁶ are H; R² is either amino, or otherwise in combination with R³ is W⁷; wherein R^{a}, R^{b}, and R^{c} in W⁷ are H; and R^{A} is selected from aliphatic groups, aromatic groups, -L-S_{c}, -L-R^{x}, and -L-R^{±}.

24. The composition of claim 21, where the compound has the formula:

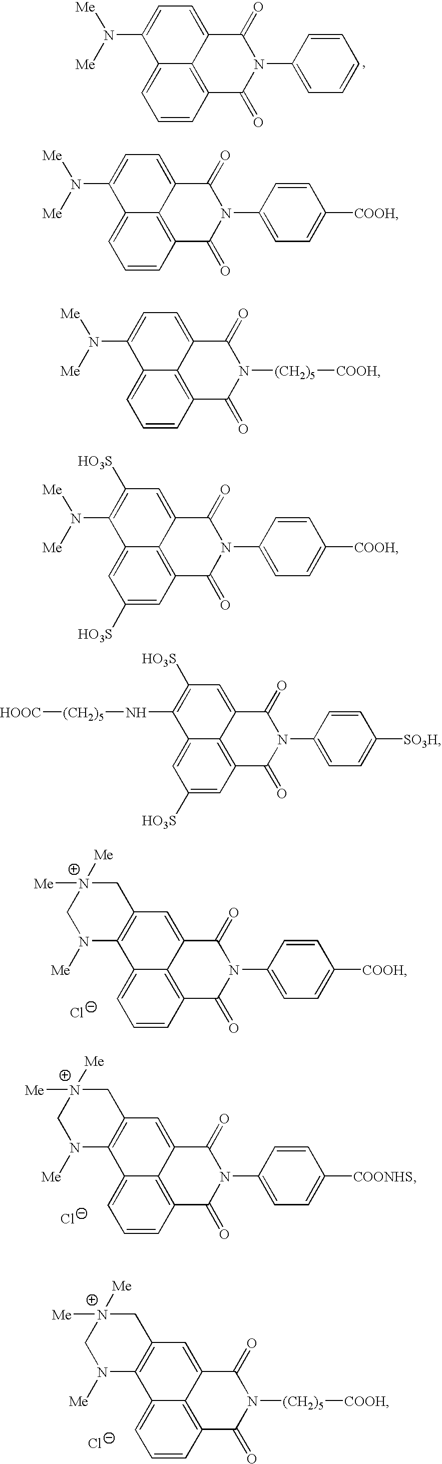

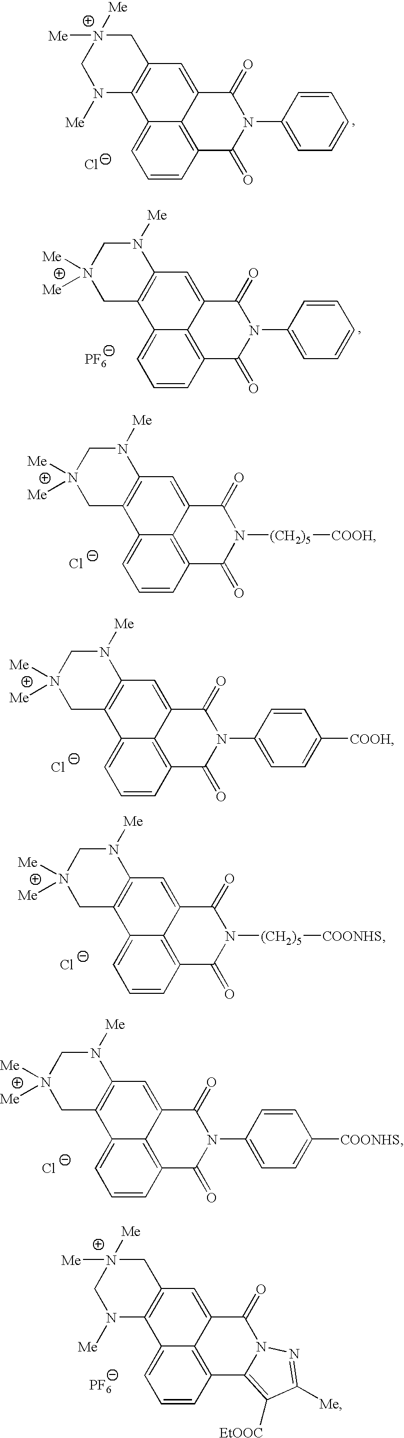

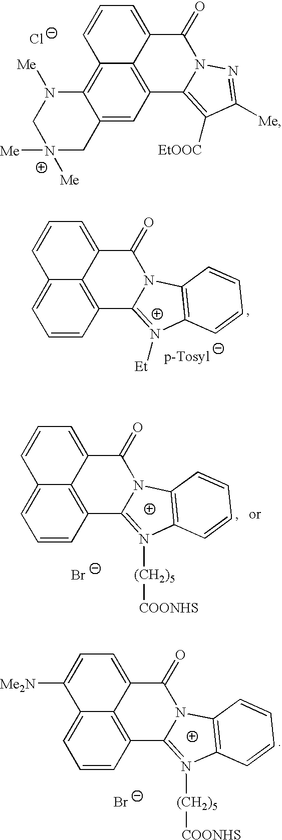

25. The composition of claim 1, where the compound has the formula 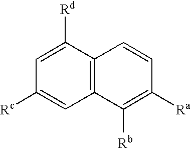
wherein R^{a} is selected from amino, NH-L-S_{c}, or NH-L-R^{x}; R^{b} is H or in combination with R^{a} forms W⁴, where Y is N or +N—R, R is -L-S_{c}, -L-R^{x}; R¹ is alkyl; R² and R³ are alkyl, -L-S_{c}, -L-R^{x} or -L-R^{±}; and R^{c} and R^{d} are sulfo.

26. The composition of claim 1, where the compound has the formula

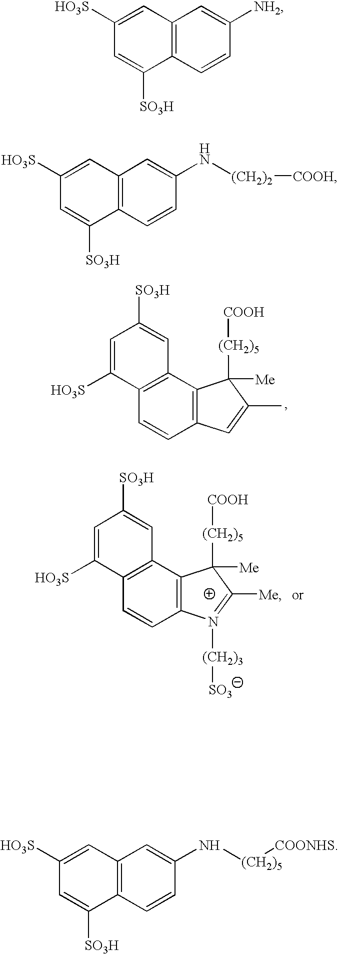

27. The composition of claim 1, where the compound includes a 1,3,3-trimethyl-1,2,3,4-tetrahydro pyrimidin-3-ium moiety W⁷ wherein each of R^{A}, R^{B} and R^{C} is methyl.

28. The composition of claim 27, where the compound has the formula

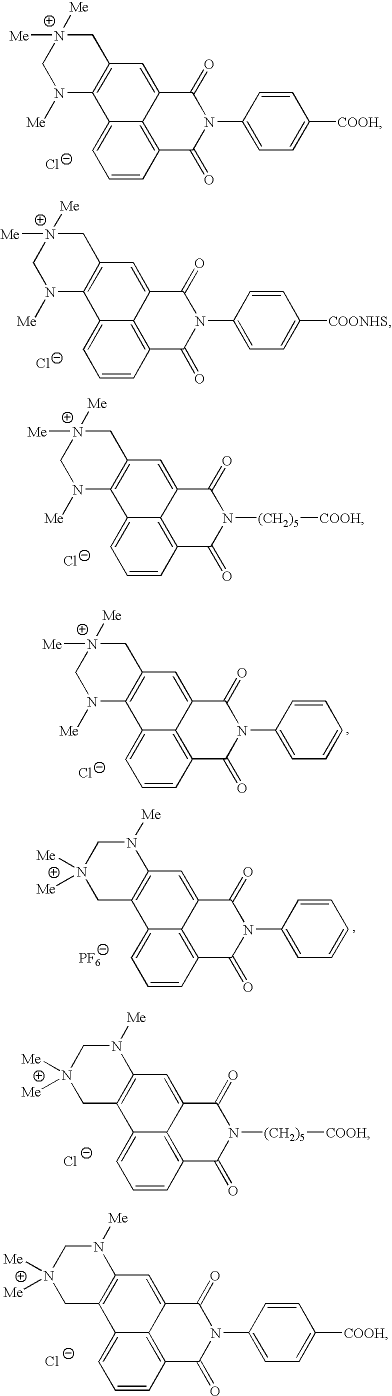

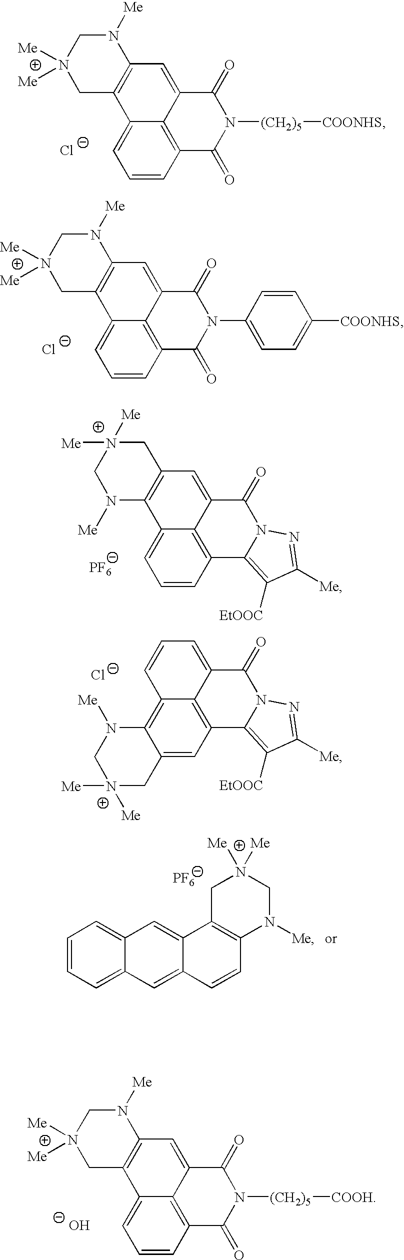

29. The composition of claim 16, further comprising a metallic nanoparticle, where the nanoparticle is configured to influence the photophysical properties of the compound at a selected distance.

30. The composition of claim 29, wherein binding between the dye-conjugate and the nanoparticle is facilitated via a specific binding pair.

31. The composition of claim 30, wherein the specific binding pair is selected from the group consisting of antigens and antibodies, ligands and receptors, biotin and streptavidin, lectin and sugar, protein A and antibodies, and oligonucleotides and complementary oligonucleotides.

32. The composition of claim 1, wherein the compound is luminescent and has a luminescence lifetime on the order of 10 nanoseconds (ns) or longer.

33. A method of performing a photoluminescence assay, the method comprising:
selecting a photoluminescent compound according to claim 1;
exciting the photoluminescent compound with excitation light; and
detecting emission light emitted by the photoluminescent compound.

34. The method of claim 33, wherein detecting emission light includes detecting fluorescence.

35. The method of claim 33, wherein detecting emission light includes detecting phosphorescence.

36. The method of claim 33, further comprising analyzing the emission light, and determining at least one of luminescence intensity, lifetime, or polarization.

37. The method of claim 33, further comprising analyzing the emission light and determining luminescence lifetime.

36. The method of claim 32, further comprising analyzing the emission light and determining luminescence polarization.

37. The method of claim 32, further comprising associating the photoluminescent compound with a second molecule.

38. The method of claim 38, where the second molecule is an energy-transfer acceptor or an energy-transfer donor.

39. The method of claim 36, further comprising performing a multi-lifetime assay, where different assay components are labeled with dyes of this invention having similar absorption and emission maxima but different luminescent lifetimes.

40. The method of claim 32, further comprising performing a cell-based assay.
